(19) United States
(12) Patent Application Publication
(10) Pub. No.: US 2026/0286361 A1
(43) Pub. Date: Sep. 24, 2026

(54) METHODS OF TREATING CHRONIC HEPATITIS B

(71) Applicant: GlaxoSmithKline Intellectual Property Development Limited, Stevenage (GB)

(72) Inventors: Leigh Michael FELTON, Stevenage (GB); Susan Amanda DIXON, Stevenage (GB); Robert Conrad ELSTON, Stevenage (GB); Dickens THEODORE, Durham, PA (US)

(21) Appl. No.: 19/474,261

(22) PCT Filed: Apr. 11, 2024
(86) PCT No.: PCT/IB2024/053559

§ 371 Date: Oct. 10, 2025

Related U.S. Application Data
(60) Provisional application No. 63/495,613, filed on Apr. 12, 2023.
(60) Provisional application No. 63/580,470, filed on Sep. 5, 2023.
(60) Provisional application No. 63/595,033, filed on Nov. 1, 2023.

(51) Int. Cl. C12N 15/113; A61K 31/712; A61K 38/21; A61K 47/60

(52) U.S. Cl. CPC C12N 15/1131; A61K 31/712; A61K 38/212; A61K 47/60; C12N 2310/11; C12N 2310/321

(57) ABSTRACT

The present disclosure relates to methods for treating a human with chronic hepatitis B. The methods comprise administering to a human in need thereof a therapeutically effective amount of antisense oligonucleotide (ASO) (e.g. bepirovirsen) and a therapeutically effective amount of interferon, wherein the human has a HBsAg baseline level of not greater than a threshold level. The present disclosure further relates to specific dosing regimens.

### FIELD

The present disclosure relates to methods for treating a human with chronic hepatitis B. These methods comprise administering to a human in need thereof a therapeutically effective amount of an antisense oligonucleotide (e.g. bepirovirsen) and a therapeutically effective amount of interferon, wherein the human has a HBsAg baseline level of not greater than a threshold level. The present disclosure further relates to specific dosing regimens.

### BACKGROUND

Hepatitis B virus (HBV) is a strict hepatotropic, partially double-stranded DNA virus. Although DNA is the genetic material, the replication cycle involves a reverse transcription step to copy a pregenomic RNA into DNA. Primary infection with HBV causes an acute hepatitis with symptoms of organ inflammation, fever, jaundice, and increased liver transaminases in blood. Those patients that are not able to overcome the acute virus infection suffer a chronic disease progression over many years with increased risk of developing cirrhotic liver or liver cancer.

HBV infection results in the production of two different particles: 1) the HBV virus itself (or Dane particle) which includes a viral capsid assembled from the HBV core antigen protein (HBcAg) and is covered by the hepatitis B surface antigen (HBsAg) and is capable of reinfecting cells and 2) subviral particles (or SVPs) which are high density lipoprotein-like particles comprised of lipids, cholesterol, cholesterol esters and the small and medium forms of the hepatitis B surface antigen (HBsAg) which are non-infectious. For each Dane particle produced, 1,000-10,000 SVPs are released into the blood. As such SVPs (and the HBsAg protein they carry) represent the overwhelming majority of viral protein in the blood. HBV infected cells also secrete a soluble proteolytic product of the pre-core protein called the HBV e-antigen (HBeAg). The presence of HBeAg in the serum of patients can serve as a marker of active replication in chronic hepatitis.

Currently the recommended therapies for chronic HBV infection by the American Association for the Study of Liver Diseases (AASLD) and the European Association for the Study of the Liver (EASL) include interferon alpha (IFN-α), pegylated interferon alpha-2a, entecavir, and tenofovir. However, typical interferon therapy is 48-weeks and is associated with tolerability issues due to side effects, and HBeAg seroconversion, 24 weeks after therapy has ceased, ranges from 27 to 36%. Seroconversion of HBsAg is even lower-approximately 3% observed immediately after treatment ceases, with an increase to upwards of 12% after 5 years.

The nucleoside and nucleotide therapies entecavir and tenofovir have been successful at reducing viral load (HBV DNA), but the rates of HBeAg seroconversion and HBsAg loss are even lower than those obtained using IFNα therapy. Other similar therapies, including lamivudine (3TC), telbivudine (LdT), and adefovir are also used, but for nucleoside/nucleotide therapies in general, the emergence of resistance limits therapeutic efficacy.

Antisense oligonucleotides (ASOs) include bepirovirsen, an experimental antisense oligonucleotide in development for the treatment of chronic hepatitis B (CHB) infection. Bepirovirsen directly targets all HBV messenger ribonucleic acids (mRNAs) via ribonuclease H (RNase H) mediated degradation, resulting in the reduction of viral proteins including HBsAg.

A Phase 2b clinical study (B-Clear) was conducted to evaluate the efficacy and safety of treatment with bepirovirsen in participants with chronic hepatitis B. See Yuen et al. “Efficacy and safety of bepirovirsen in chronic hepatitis B infection,” N. Engl. J. Med. 2022; 387:1957-68. Bepirovirsen at a dose of 300 mg per week for 24 weeks resulted in sustained HBsAg and HBV DNA loss in 9 to 10% of participants with chronic HBV infection.

There is still a need to discover and develop new anti-HBV therapies, more particularly, combination therapies capable of achieving a greater functional cure rate for patients with chronic hepatitis B (CHB).

### SUMMARY

In a first aspect of the invention, a method for treating chronic hepatitis B (CHB) in a human in need thereof is provided, the method comprising administering to the human a therapeutically effective amount of an antisense oligonucleotide (ASO) and a therapeutically effective amount of interferon, wherein the human has a HBsAg baseline level of not greater than a threshold level.

In a second aspect of the invention, a method for treating chronic hepatitis B (CHB) in a human in need thereof is provided, the method comprising administering to the human a therapeutically effective amount of bepirovirsen and a therapeutically effective amount of interferon, wherein the human has a HBsAg baseline level of not greater than a threshold level.

In a third aspect of the invention, a method for treating CHB in a human in need thereof is provided, the method comprising:
(a) determining that the human has a HBsAg baseline level of not greater than a threshold level; and
(b) administering to the human a therapeutically effective amount of an ASO (e.g. bepirovirsen) and a therapeutically effective amount of interferon.

In a fourth aspect of the invention, a method for treating CHB in a human in need thereof is provided, the method comprising:
(a) measuring the HBsAg baseline level of the human;
(b) comparing the HBsAg baseline level with a threshold level; and
(c) if the HBsAg baseline level is not greater than the threshold level, then administering to the human a therapeutically effective amount of an ASO (e.g. bepirovirsen) and a therapeutically effective amount of interferon.

In a fifth aspect of the invention, a combination of an ASO (e.g. bepirovirsen) and PegIFN alpha-2a for the treatment of CHB in a human is provided, wherein:
(a) the human to be treated has a baseline HBsAg≤3000 IU/mL;
(b) the ASO is to be administered to the human weekly at a dose of about 300 mg for 12 weeks or 24 weeks; and
(c) PegIFN alpha-2a is to be administered to the human weekly at a dose of about 180 μg for 12 weeks to 48 weeks following cessation of the ASO.

In a sixth aspect of the invention, a combination of an ASO (e.g. bepirovirsen) and PegIFN alpha-2a for achieving a functional HBV cure in a human with CHB is provided, wherein:
(a) the human to be treated has a baseline HBsAg≤3000 IU/mL;
(b) the ASO is to be administered to the human weekly at a dose of about 300 mg for 12 weeks or 24 weeks; and
(c) PegIFN alpha-2a is to be administered to the human weekly at a dose of about 180 μg for 12 weeks to 48 weeks following cessation of the ASO.

In a seventh aspect of the invention, a use of an ASO (e.g. bepirovirsen) in the manufacture of a medicament for the treatment of CHB in a human is provided, wherein:
(a) the human to be treated has a baseline HBsAg≤3000 IU/mL;
(b) the ASO is to be administered to the human weekly at a dose of about 300 mg for 12 weeks or 24 weeks; and
(c) PegIFN alpha-2a is to be administered weekly to the human at a dose of about 180 μg for 12 weeks to 48 weeks following cessation of the ASO.

In an eighth aspect of the invention, a use of PegIFN alpha-2a in the manufacture of a medicament for the treatment of CHB in a human is provided, wherein:
(a) the human to be treated has a baseline HBsAg≤3000 IU/mL;
(b) an ASO is to be administered to the human weekly at a dose of about 300 mg for 12 weeks or 24 weeks; and
(c) PegIFN alpha-2a is to be administered to the human weekly at a dose of about 180 μg for 12 weeks to 48 weeks following cessation of the ASO.

### BRIEF DESCRIPTION OF THE DRAWINGS

FIG. 1 depicts the study design of a Phase 2b clinical trial to assess efficacy and safety of treatment with bepirovirsen and PegIFN in patients with CHB (B-Together Study).

FIG. 2 depicts the proportion of participants with HBsAg<0.05 IU/mL and HBV DNA<LLOQ in the absence of newly initiated antiviral therapy (rescue therapy) at the end of study, overall and by baseline HBsAg level (B-Together Study).

FIG. 3 depicts the study design of a Phase 3b clinical trial to assess the efficacy and safety of treatment with bepirovirsen and PegIFN sequential therapy in nucleos(t)ide analogue-treated patients with CHB (B-Sustained Study).

### DETAILED DESCRIPTION

### Definitions

Unless defined otherwise, all technical and scientific terms used herein have the same meanings as commonly understood by one of ordinary skill in the art. For example, certain terms used herein are defined as described in “A multilingual glossary of biotechnological terms: (IUPAC Recommendations),” Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise,” and variations such as “comprises” and “comprising,” will be understood to imply the inclusion of a stated element, integer, or step, or group of elements, integers, or steps but not the exclusion of any other element, integer, or step, or group of elements, integers, or steps.

Unless otherwise indicated, the following terms have the following meanings:

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass±10% of the recited value and the range is included.

“Antisense oligonucleotide (ASO)” refers to a single-stranded oligonucleotide having a nucleobase sequence that permits hybridization to a corresponding region or segment of a target nucleic acid.

“Baseline” values of certain substances (e.g., HBsAg, HBeAg, HBsAb, HBeAb, HBV DNA, ALT) are measured in the blood samples taken from patients before the first dose of treatment.

Bepirovirsen is an ASO currently in clinical evaluation for treating Chronic HBV infections. It is compound ISIS No. 505358 as disclosed in WO2012/145697. Bepirovirsen has 20 linked nucleosides and has a nucleobase sequence of 5′-GCAGAGGTGAAGCGAAGTGC-3′ (SEQ ID NO:1), and it includes:
a gap segment consisting of ten linked deoxynucleosides,
a 5′ wing segment consisting of 5 linked nucleosides, and
a 3′ wing segment consisting of 5 linked nucleosides,
wherein the gap segment is positioned between the 5′ wing segment and the 3′ wing segment, wherein each nucleoside of each wing segment includes a 2′-O-methoxyethyl sugar, wherein each internucleoside linkage is a phosphorothioate linkage, and wherein each cytosine is a 5-methylcytosine. The CAS Registry Number of bepirovirsen is 1403787-62-1.

AHB-137 is an unconjugated ASO that is believed to have the same nucleobase sequence as bepirovirsen. AHB-137 is developed by Ausper Biopharma Co., Ltd. and is currently investigated in clinical studies for treating HBV infections. See WO2023/131098; Qiu, X. et al., “AHB-137, a novel and potent hepatitis B virus antisense oligonucleotide with a favorable preclinical pharmacokinetics and safety profile,” EASL 2023, Abstract No. SAT-178 (concluding that “the novel HBV ASO AHB-137 has demonstrated a favorable preclinical pharmacokinetics and safety profile”); Wu, X. et al., “AHB-137, a novel hepatitis B virus antisense oligonucleotide with substantially enhanced in vitro and in vivo antiviral activity,” EASL 2023, Abstract No. SAT-173 (concluding that “AHB-137 has more potent activity in reducing serum HBsAg than BPV [bepirovirsen] across multiple in vitro and in vivo HBV models, by reducing intrahepatic HBV RNA and HBsAg”).

“Bicyclic sugar” means a furanose ring modified by the bridging of two non-geminal carbon atoms. A bicyclic sugar is a modified sugar.

“Chronic hepatitis B (CHB) infection” occurs when a person initially suffers from an acute infection but is then unable to fight off the infection. About 90% of infants infected at birth will progress to chronic disease. However, as a person ages, the risk of chronic infection decreases such that between 20%-50% of people infected as children and less than 10% of people infected as adults will progress from acute to chronic infection. The terms “chronic hepatitis B infection”, “chronic hepatitis B viral infection”, “chronic hepatitis B”, “chronic HBV infection”, “CHB infection” and “CHB” are used interchangeably herein.

“Dose” refers to a specified quantity of an active pharmaceutical agent provided in a single administration, or in a specified time period. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

“Dosing regimen” is a combination of doses designed to achieve one or more desired effects.

“Duration” refers to the period of time during which an activity or event continues. In certain embodiments, the duration of treatment is the period of time during which doses of a pharmaceutical agent are administered.

“Functional cure” refers to sustained suppression (24 weeks or longer) of HBV DNA (<lower limit of quantification or “LLOQ”) off all HBV treatment with HBsAg loss (<0.05 IU/mL) or HBsAg negative with or without HBsAb after a finite duration of therapy.

“HBV” refers to mammalian hepatitis B virus, including human hepatitis B virus. The term encompasses geographical genotypes of hepatitis B virus, particularly human hepatitis B virus, as well as variant strains of geographical genotypes of hepatitis B virus. Human geographical genotypes of HBV include, but are not limited to, genotypes: A (Northwest Europe, North America, Central America); B (Indonesia, China, Vietnam); C (East Asia, Korea, China, Japan, Polynesia, Vietnam); D (Mediterranean area, Middle East, India); E (Africa); F (Native Americans, Polynesia); G (United States, France); and H (Central America).

“HBV antigen” refers to any hepatitis B virus antigen or protein, including core proteins such as “hepatitis B core antigen” or “HBcAg,” “hepatitis B E antigen” or “HBeAg,” and envelope proteins such as “HBV surface antigen” or “HBsAg.”

“Hepatitis B E antigen” or “HBeAg” is a secreted, non-particulate form of HBV core protein. HBV antigens HBeAg and HBcAg share primary amino acid sequences, so show cross-reactivity at the T cell level. HBeAg is not required for viral assembly or replication, although studies suggest they may be required for establishment of chronic infection.

“HBV surface antigen,” “HBsAg,” or “HBs” is the envelope protein of infectious HBV viral particles (Dane Particles), but it is also secreted as a non-infectious particle (subviral particle, SVP) with serum levels 1000-fold higher than HBV viral particles. The serum levels of HBsAg in an infected person or animal can be as high as 1000 μg/mL (Kann and Gehrlich (1998) Topley & Wilson's Microbiology and Microbial Infections, 9th ed. 745).

“HBV-related condition” refers to any disease, biological condition, medical condition, or event which is exacerbated, caused by, related to, associated with, or traceable to a hepatitis B virus infection, exposure, or illness. The term hepatitis B-related condition includes chronic HBV infection, inflammation, fibrosis, cirrhosis, liver cancer, serum hepatitis, jaundice, liver inflammation, liver fibrosis, liver cirrhosis, liver failure, diffuse hepatocellular inflammatory disease, hemophagocytic syndrome, HBV viremia, liver disease related to transplantation, and conditions having symptoms which may include any or all of the following: flu-like illness, weakness, aches, headache, fever, loss of appetite, diarrhea, nausea and vomiting, pain over the liver area of the body, clay- or grey-colored stool, itching all over, and dark-colored urine, when coupled with a positive test for presence of a hepatitis B virus, a hepatitis B viral antigen, or a positive test for the presence of an antibody specific for a hepatitis B viral antigen.

“Induce,” “inhibit,” “potentiate,” “elevate,” “increase,” “decrease” or the like, generally denote quantitative differences between two states. Such terms may refer to a statistically significant difference between the two states. For example, “an amount effective to inhibit the activity or expression of HBV” refers to the level of activity or expression of HBV in a treated cell and this will quantitatively differ, and may be statistically significant, from the level of HBV activity or expression in untreated cells. Such terms are applied to, for example, levels of expression and levels of activity.

“Modified internucleoside linkage” refers to a substitution or any change from a naturally occurring internucleoside bond (i.e. a phosphodiester internucleoside bond).

“Modified nucleobase” means any nucleobase other than adenine, cytosine, guanine, thymidine, or uracil.

“Modified sugar” means substitution and/or any change from a natural sugar moiety.

“Nucleobase” refers to a heterocyclic moiety capable of pairing with a base of another nucleic acid.

“Nucleobase sequence” refers to the order of contiguous nucleobases independent of any sugar, linkage, and/or nucleobase modification.

“Interferon” is used herein to mean interferon suitable for the treatment of chronic hepatitis B in a human; this includes interferon alpha and interferon lambda and pegylated forms thereof (see Ye et al., Front. Immunol. 2021, 12:733364).

“PegIFN”, “peginterferon”, “pegylated interferon”, and “pegylated interferon alpha”, unless otherwise indicated, are used interchangeably herein and refer to a pegylated form of human interferon alpha. PegIFN include alpha-2a and PegIFN alpha-2b. PEG-IFN alpha-2a is synthesized by the covalent attachment of a branched bis-[monomethoxy polyethylene glycol (PEG)] polymer, with a molecular mass of about 44000, to interferon alfa-2a, which is produced using recombinant DNA technology. PegIFN alpha-2a is marketed under the brand name PEGASYS. PegIFN alpha-2b include PEGINTRON and PEGBERON.

“Pharmaceutically acceptable salts” refer to physiologically acceptable salts of compounds, i.e., salts that retain the desired biological activity of the parent active ingredients and do not impart undesired toxicological effects thereto.

“Phosphorothioate linkage” refers to a linkage between nucleotides where the phosphodiester bond is modified by replacing one of the non-bridging oxygen atoms with a sulfur atom. A phosphorothioate linkage is a modified internucleoside linkage.

“Region” is defined as a portion of, for example, a nucleic acid having at least one identifiable structure, function, or characteristic.

“Stable nucleoside or nucleotide analogue (NA) therapy” is defined as no changes to the nucleoside or nucleotide analogue regimen for at least 6 months prior to the treatment disclosed in the present disclosure and with no planned changes to the regimen for the duration of the treatment.

“Segments” may refer to smaller, or sub-portions of, regions within, for example, a nucleic acid.

“Seroclearance” refers to the clearance or removal of an antigen (like HBsAg and/or HBV DNA levels) from the blood (or a measurement below the lower limit of quantification (i.e. <LLOQ) by the lab) in a CHB patient following treatment. In testing human samples, when serum HBsAg level is measured by a sandwich immunoassay with COBAS HBsAg quant II (Roche), the LLOQ is 0.05 IU/mL. In testing human samples, when the serum HBV DNA level is measured with COBAS Ampliprep/COBAS Taqman HBV test v.2.0 (Roche), the LLOQ is 20 IU/mL. When a qualitative assay is used to measure the serum HBsAg level, a binary terminology (positive/detected, negative/undetected) is used.

“Subject” refers to a human or non-human animal selected for treatment or therapy. In one embodiment, the subject is a human.

“Therapeutically effective amount” refers to the administration of a pharmaceutical agent to a subject, either alone or as part of a pharmaceutical composition and either in a single dose or as part of a series of doses, in an amount capable of having any detectable, positive effect on any symptom, aspect, or characteristic of a disease or condition when administered to the subject.

“Treatment” refers to administering a pharmaceutical agent to a subject to affect an alteration or improvement of the disease or condition. The term “treating” as used herein in relation to chronic hepatitis B infection refers to the administration of suitable compositions with the intention of reducing the symptoms of CHB, preventing the progression of CHB or reducing the level of one or more detectable markers of CHB.

### Methods

The present disclosure provides a method for treating chronic hepatitis B (CHB) in a human subject in need thereof, the method comprising administering to the subject a therapeutically effective amount of an antisense oligonucleotide (ASO) (e.g. bepirovirsen) and a therapeutically effective amount of interferon, wherein the subject has a HBsAg baseline level of not greater than a threshold level.

In some embodiments, the ASO comprises 20 linked nucleosides and has a nucleobase sequence of SEQ ID NO:1. In some embodiments, the ASO consists of 20 linked nucleosides and has a nucleobase sequence of SEQ ID NO: 1. In some embodiments, the ASO comprises at least one modified sugar. In some embodiments, the at least one modified sugar is a bicyclic sugar or comprises a 2′-O-methoxyethyl(2′-MOE) group.

In some embodiments, the ASO comprises at least one modified internucleoside linkage. In some embodiments, the at least one modified internucleoside linkage is a phosphorothioate linkage. In some embodiments, each internucleoside linkage of the ASO is a phosphorothioate linkage.

In some embodiments, the ASO comprises at least one modified nucleobase. In some embodiments, the modified nucleobase is 5-methylcytosine.

In some embodiments, the ASO comprises:
a gap segment consisting of linked deoxynucleosides,
a 5′ wing segment consisting of linked nucleosides, and
a 3′ wing segment consisting of linked nucleosides,

wherein the gap segment is positioned between the 5′ wing segment and the 3′ wing segment and wherein each nucleoside of each wing segment comprises a modified sugar.

In some embodiments, the ASO comprises a separator segment placed in between gap segments as disclosed in WO2023/131098, which is incorporated herein by reference in its entirety. In one embodiment, the ASO is selected from Compounds AUS1233/AUS1138, AUS1444, AUS1458, AUS1459, AUS1460, AUS1427/AUS1461, AUS1462, AUS1463, AUS1464, AUS1465, AUS1463, AUS1467, AUS1468, AUS1470, AUS1471, AUS1472, AUS1473, AUS1474, AUS1475, AUS1476/AUS1493, AUS1478, AUS1479, AUS1489, AUS1490, AUS1443, and AUS1322 as disclosed in WO2023/131098. In one embodiment, the ASO is selected from Compounds AUS1233/AUS1138, AUS1463, and AUS1476/AUS1493 as disclosed in WO2023/131098. In one embodiment, the ASO is Compound AUS1493 as disclosed in WO2023/131098.

In one embodiment, the ASO is bepirovirsen. In another embodiment, the ASO is AHB-137.

In another aspect, the present disclosure provides a method for treating chronic hepatitis B in a human in need thereof, the method comprising:
(a) determining that the human has a HBsAg baseline level of not greater than a threshold level; and
(b) administering to the human a therapeutically effective amount of an ASO (e.g. bepirovirsen) and a therapeutically effective amount of interferon.

The HBsAg baseline level in the blood of a CHB patient is measured by methods known in the art before the start of treatment. As used herein, the step “determining” can be achieved by measuring the HBsAg baseline level of the patient and comparing the baseline level with a predetermined threshold level. The step “determining” can also be achieved by receiving the HBsAg baseline level information from the patient or a testing facility and comparing the HBsAg baseline level with a predetermined threshold level.

In another aspect, the present disclosure provides a method for treating chronic hepatitis B in a human in need thereof, the method comprising:
(a) measuring the HBsAg baseline level of the human;
(b) comparing the HBsAg baseline level with a threshold level; and
(c) if the HBsAg baseline level is not greater than the threshold level, then administering to the human a therapeutically effective amount of an ASO (e.g. bepirovirsen) and a therapeutically effective amount of interferon.

It was reported that a low HBsAg baseline level can be used as a predictor of HBsAg loss in HBeAg-negative patients treated with PegIFN and adefovir. See Takkenberg, et al., “Baseline hepatitis B surface antigen (HBsAg) as predictor of sustained HBsAg loss in chronic hepatitis B patients treated with pegylated interferon-α2a and adefovir,” Antivir. Ther. 2013; 18(7):895-904. In particular, a HBsAg baseline level below 400 IU/mL was identified as a good predictor for treating HBeAg-negative patients with PegIFN and adefovir. However, a low HBsAg baseline level could not predict HBsAg loss in HBeAg-positive patients in the same study.

In the methods as disclosed herein, the ASO (e.g. bepirovirsen) can be administered as a free acid, a pharmaceutically acceptable salt thereof (e.g., a sodium salt), or a combination thereof. In some embodiments, the ASO (e.g. bepirovirsen) is administered as a free acid. In some embodiments, the ASO (e.g. bepirovirsen) is administered as a pharmaceutically acceptable salt thereof (e.g., a sodium salt). In some embodiments, the ASO (e.g. bepirovirsen) is administered as a combination of a free acid and a sodium salt. In some embodiments, the ASO (e.g. bepirovirsen) is administered by subcutaneous injection.

As used herein, the “therapeutically effective amount of the ASO (e.g. bepirovirsen)” refers to the amount of the ASO (e.g. bepirovirsen) free acid. In some embodiments, the therapeutically effective amount of the ASO (e.g. bepirovirsen) is about 150 mg to 450 mg once weekly. In some embodiments, the therapeutically effective amount of the ASO (e.g. bepirovirsen) is about 150 mg, 200 mg, 250 mg, 300 mg, 350 mg, 400 mg, or 450 mg once weekly, or in a range between any two preceding values. In some embodiments, the therapeutically effective amount of the ASO (e.g. bepirovirsen) is about 150 mg once weekly. In some embodiments, the therapeutically effective amount of the ASO (e.g. bepirovirsen) is about 300 mg once weekly. In some embodiments, the ASO (e.g. bepirovirsen) is administered weekly with additional loading doses in the first two weeks on Day 4 and Day 11 following the first dose (also referred to as “2 loading doses”). In some embodiments, the ASO (e.g. bepirovirsen) is administered at a dose of about 300 mg once weekly with additional loading doses in the first two weeks on Day 4 and Day 11 following the first dose. In a particular embodiment, the loading dose is 300 mg.

In some embodiments, the ASO (e.g. bepirovirsen) is administered for about 12 to 48 weeks. In some embodiments, the ASO (e.g. bepirovirsen) is administered for 12 weeks, 24 weeks, or 48 weeks, or for a range between any two preceding periods. In one embodiment, the ASO (e.g. bepirovirsen) is administered for 12 weeks. In one embodiment, the ASO (e.g. bepirovirsen) is administered for 24 weeks. In one embodiment, the ASO (e.g. bepirovirsen) is administered for 48 weeks. In one embodiment, the ASO (e.g. bepirovirsen) is administered for 12 weeks or 24 weeks, with additional loading doses on Day 4 and Day 11 following the first dose.

In some embodiments, the ASO (e.g. bepirovirsen) is administered at a dose of about 300 mg once weekly for 24 weeks. In some embodiments, the ASO (e.g. bepirovirsen) is administered at a dose of about 300 mg once weekly for 12 weeks. In some embodiments, the ASO (e.g. bepirovirsen) is administered at a dose of about 300 mg once weekly for 12 weeks, and then at a dose of about 150 mg once weekly for 12 weeks.

In some embodiments, the ASO (e.g. bepirovirsen) is administered at a dose of about 300 mg once weekly for 24 weeks, with additional loading doses each of 300 mg, on Day 4 and Day 11 following the first dose. In some embodiments, the ASO (e.g. bepirovirsen) is administered at a dose of about 300 mg once weekly for 12 weeks, with additional loading doses each of 300 mg on Day 4 and Day 11 following the first dose.

In some embodiments, the present disclosure provides a method for treating CHB in a human in need thereof comprising administering to the human a therapeutically effective amount of an ASO (e.g. bepirovirsen) and a therapeutically effective amount of interferon, wherein the subject has a HBsAg baseline level of not greater than a threshold level. In some embodiments, the threshold level of the HBsAg baseline is in the range of about 1000 IU/mL to 3,000 IU/mL.

In some embodiments, the threshold level of the HBsAg baseline is about 1000, 1500, 2000, 2500, or 3000 IU/mL, or in a range between any two of the preceding numbers. In some embodiments, the threshold level of the HBsAg baseline is about 1000, 2000, or 3000 IU/mL. In some embodiments, the threshold level of the HBsAg baseline is about 1000 IU/mL. In some embodiments, the threshold level of the HBsAg baseline is about 3000 IU/mL.

In one embodiment, the present disclosure provides a method for treating CHB in a human in need thereof, the method comprising administering to the human a therapeutically effective amount of an ASO (e.g. bepirovirsen) and a therapeutically effective amount of interferon, wherein the human has a HBsAg baseline level of not greater than about 1000 IU/mL. In one embodiment, the present disclosure provides a method for treating CHB in a human in need thereof, the method comprising administering to the human a therapeutically effective amount of an ASO (e.g. bepirovirsen) and a therapeutically effective amount of interferon, wherein the human has a HBsAg baseline level of not greater than about 3000 IU/mL. In an embodiment, the interferon is interferon alpha or interferon lambda. In another embodiment, the interferon is PegIFN.

In one embodiment, the present disclosure provides a method for treating CHB in a human in need thereof, the method comprising administering an ASO (e.g. bepirovirsen) to the human at a dose of 300 mg once weekly for 24 weeks, wherein the human has a HBsAg baseline level of not greater than about 1000 IU/mL. In one embodiment, the present disclosure provides a method for treating CHB in a human in need thereof, the method comprising administering an ASO (e.g. bepirovirsen) to the human at a dose of 300 mg once weekly for 24 weeks, wherein the human has a HBsAg baseline level of not greater than about 3000 IU/mL.

In one embodiment, the present disclosure provides a method for treating CHB in a human in need thereof, the method comprising administering an ASO (e.g. bepirovirsen) to the human at a dose of 300 mg once weekly for 24 weeks, wherein the human has a HBsAg baseline level of not greater than about 1000 IU/mL and is HBeAg negative. In one embodiment, the present disclosure provides a method for treating CHB in a human in need thereof, the method comprising administering an ASO (e.g. bepirovirsen) to the human at a dose of 300 mg once weekly for 24 weeks, wherein the human has a HBsAg baseline level of not greater than about 3000 IU/mL and is HBeAg negative.

In one embodiment, the present disclosure provides a method for treating CHB in a human in need thereof, the method comprising administering an ASO (e.g. bepirovirsen) to the human at a dose of 300 mg once weekly for 24 weeks, wherein the human has a HBsAg baseline level of not greater than about 1000 IU/mL and is HBeAg negative, and wherein the human is on stable nucleoside or nucleotide analogue (NA) therapy. In one embodiment, the present disclosure provides a method for treating CHB in a human in need thereof, the method comprising administering an ASO (e.g. bepirovirsen) to the human at a dose of 300 mg once weekly for 24 weeks, wherein the human has a HBsAg baseline level of not greater than about 3000 IU/mL and is HBeAg negative, and wherein the human is on stable nucleoside or nucleotide analogue (NA) therapy.

In an embodiment, the ASO (e.g. bepirovirsen) and interferon are administered sequentially. In an embodiment the ASO (e.g. bepirovirsen) is administered first and interferon is administered second. In an embodiment, the human is assessed for interferon eligibility before the interferon treatment based on the label. In an embodiment, interferon is administered following the ASO (e.g. bepirovirsen) cessation. In an embodiment, interferon is interferon alpha or interferon lambda. In another embodiment, interferon is PegIFN. In one embodiment, interferon is PegIFN alpha-2a. In one embodiment, interferon is PegIFN alpha-2b.

In an embodiment, the HBsAg level in a human with CHB infection is reduced from baseline following the ASO (e.g. bepirovirsen) treatment and prior to interferon, in particular PegIFN alpha-2a, treatment. In an embodiment, the HBsAg level in the human with CHB infection is below the lower limit of quantification (LLOQ) following the ASO (e.g. bepirovirsen) treatment and prior to interferon, in particular PegIFN alpha-2a, treatment.

In another aspect of the invention, a combination of an ASO (e.g. bepirovirsen) and interferon, in particular PegIFN alpha-2a, is provided for the treatment of CHB in a human, wherein: (a) the human to be treated has a baseline HBsAg≤3000 IU/mL; (b) the ASO (e.g. bepirovirsen) is to be administered to the human weekly at a dose of about 300 mg for 12 weeks or 24 weeks; and (c) interferon is to be administered weekly to the human at a dose of about 180 μg for 12 weeks to 48 weeks following cessation of the ASO (e.g. bepirovirsen). In an embodiment, the human to be treated has a baseline HBsAg≤1000 IU/mL. In an embodiment, the HBsAg level in the human with CHB is below the LLOQ following the ASO (e.g. bepirovirsen) treatment and prior to interferon treatment. In an embodiment, the ASO (e.g. bepirovirsen) is to be administered to the human weekly at a dose of about 300 mg for 12 weeks. In an embodiment, the ASO (e.g. bepirovirsen) is to be administered to the human weekly at a dose of about 300 mg for 24 weeks. In an embodiment, PegIFN alpha-2a is to be administered weekly to the human at a dose of about 180 μg for 12 weeks. In an embodiment, PegIFN alpha-2a is to be administered weekly to the human at a dose of about 180 μg for 24 weeks. In an embodiment, PegIFN alpha-2a is to be administered weekly to the human at a dose of about 180 μg for 48 weeks.

In an aspect of the invention, a combination of an ASO (e.g. bepirovirsen) and interferon, in particular PegIFN alpha-2a, is provided for achieving a functional HBV cure in a human with CHB, wherein: (a) the human to be treated has a baseline HBsAg≤3000 IU/mL; (b) the ASO (e.g. bepirovirsen) is to be administered to the human weekly at a dose of about 300 mg for 12 weeks or 24 weeks; and (c) interferon is to be administered weekly to the human at a dose of about 180 μg for 12 weeks to 48 weeks following cessation of the ASO (e.g. bepirovirsen). In an embodiment, the human to be treated has a baseline HBsAg≤1000 IU/mL. In an embodiment, the HBsAg level in the human with CHB is below the LLOQ following the ASO (e.g. bepirovirsen) treatment and prior to interferon treatment. In an embodiment, the ASO (e.g. bepirovirsen) is to be administered to the human weekly at a dose of about 300 mg for 12 weeks. In an embodiment, the ASO (e.g. bepirovirsen) is to be administered to the human weekly at a dose of about 300 mg for 24 weeks. In an embodiment, PegIFN alpha-2a is to be administered weekly to the human at a dose of about 180 μg for 12 weeks. In an embodiment, PegIFN alpha-2a is to be administered weekly to the human at a dose of about 180 μg for 24 weeks. In an embodiment, PegIFN alpha-2a is to be administered weekly to the human at a dose of about 180 μg for 48 weeks.

In some embodiments, the functional cure rate is greater than 15%, greater than 20%, greater than 25%, greater than 30%, greater than 35% or greater than 40% in patients with a baseline HBsAg≤3000 IU/mL. In some embodiments, the functional cure rate is greater than 30% in patients with a baseline HBsAg≤3000 IU/mL. In some embodiments, the functional cure rate is greater than 35% in patients with a baseline HBsAg≤3000 IU/mL. In some embodiments, the functional cure rate is greater than 40% in patients with a baseline HBsAg≤3000 IU/mL. In some embodiments, the functional cure rate is greater than 35% in patients with a baseline HBsAg≤1000 IU/mL. In some embodiments, the functional cure rate is greater than 40% in patients with a baseline HBsAg≤1000 IU/mL. In some embodiments, the functional cure rate is greater than 45% in patients with a baseline HBsAg≤1000 IU/mL. In some embodiments, the functional cure rate is greater than 50% in patients with a baseline HBsAg≤1000 IU/mL. In some embodiments, the functional cure rate is greater than 55% in patients with a baseline HBsAg≤1000 IU/mL.

In an embodiment, the human administered the combination of the ASO (e.g. bepirovirsen) and interferon is on stable NA therapy. In an embodiment, the NA therapy is lamivudine, adefovir, adefovir dipivoxil, telbivudine, entecavir, tenofovir, tenofovir disoproxil fumarate, or tenofovir alafenamide, or a pharmaceutically acceptable salt of any of the foregoing. In an embodiment, the human discontinues NA therapy after interferon cessation. In an embodiment, the human discontinues NA therapy 12 weeks after interferon cessation. In an embodiment, the human discontinues NA therapy 24 weeks after interferon cessation.

In an embodiment, the human is administered the ASO (e.g. bepirovirsen) during weeks 1-12, followed by interferon during weeks 13-36 and NA throughout weeks 1-60.

In another embodiment, the human is administered the ASO (e.g. bepirovirsen) during weeks 1-24, followed by interferon during weeks 25-48 and NA throughout weeks 1-72.

In an aspect of the invention, a use of an ASO (e.g. bepirovirsen) in the manufacture of a medicament for the treatment of CHB in a human is provided, wherein: (a) the human to be treated has a baseline HBsAg≤3000 IU/mL; (b) the ASO (e.g. bepirovirsen) is to be administered to the human weekly at a dose of about 300 mg for 12 weeks or 24 weeks; and (c) interferon, in particular PegIFN, is to be administered to the human weekly at a dose of about 180 μg for 12 weeks to 48 weeks following cessation of the ASO (e.g. bepirovirsen). In an embodiment, the human to be treated has a baseline HBsAg≤1000 IU/mL. In an embodiment, the HBsAg level in the human with CHB is below the LLOQ following the ASO (e.g. bepirovirsen) treatment and prior to interferon treatment. In an embodiment, the ASO (e.g. bepirovirsen) is to be administered to the human weekly at a dose of about 300 mg for 12 weeks. In an embodiment, the ASO (e.g. bepirovirsen) is to be administered to the human weekly at a dose of about 300 mg for 24 weeks. In an embodiment, interferon is to be administered weekly to the human at a dose of about 180 μg for 12 weeks. In an embodiment, interferon is to be administered weekly to the human at a dose of about 180 μg for 24 weeks. In an embodiment, interferon is to be administered weekly to the human at a dose of about 180 μg for 48 weeks.

In a further aspect of the invention, use of interferon, in particular PegIFN, in the manufacture of a medicament for the treatment of CHB in a human is provided, wherein: (a) the human to be treated has a baseline HBsAg≤3000 IU/mL; (b) an ASO (e.g. bepirovirsen) is to be administered to the human weekly at a dose of about 300 mg for 12 weeks or 24 weeks; and (c) interferon is to be administered to the human weekly at a dose of about 180 μg for 12 weeks to 48 weeks following cessation of the ASO (e.g. bepirovirsen). In an embodiment, the human to be treated has a baseline HBsAg≤1000 IU/mL. In an embodiment, the HBsAg level in the human with CHB is below the LLOQ following the ASO (e.g. bepirovirsen) treatment and prior to interferon treatment. In an embodiment, the ASO (e.g. bepirovirsen) is to be administered to the human weekly at a dose of about 300 mg for 12 weeks. In an embodiment, the ASO (e.g. bepirovirsen) is to be administered to the human weekly at a dose of about 300 mg for 24 weeks. In an embodiment, interferon is to be administered weekly to the human at a dose of about 180 μg for 12 weeks. In an embodiment, interferon is to be administered weekly to the human at a dose of about 180 μg for 24 weeks. In an embodiment, interferon is to be administered weekly to the human at a dose of about 180 μg for 48 weeks.

In some embodiments, the interferon is interferon alpha or interferon lambda. In some embodiments, the interferon is PegIFN. In some embodiments, PegIFN is PEGASYS. In some embodiments, PegIFN is PEGINTRON or PEGBERON. In some embodiments, the human is not treated with another HBsAg reducing agent or another immunomodulating agent. That is, the ASO (e.g. bepirovirsen) and interferon are used as a sequential dual therapy for treating CHB. A HBsAg reducing agent can be a small/short interfering RNA (siRNA), a second ASO, a nucleic acid polymer (NAP), an HBV RNA destabilizer, an HBV specific neutralizing mAb, or a combination thereof. An immunomodulating agent can be a therapeutic vaccine, a PD-1/PD-L1 inhibitor, a TLR7 agonist, a TLR8 agonist, a TLR9 agonist, or a combination thereof. In some embodiments, the human is treated with another HBsAg reducing agent or another immunomodulating agent.

In one embodiment, the human is on stable nucleoside or nucleotide analogue (NA) therapy (e.g., tenofovir disoproxil, tenofovir alafenamide, or entecavir). In some embodiments, the NA therapy is lamivudine, adefovir, adefovir dipivoxil, telbivudine, entecavir, tenofovir, tenofovir disoproxil fumarate (TDF), or tenofovir alafenamide (TAF), or a pharmaceutically acceptable salt thereof. In some embodiments, the NA therapy is entecavir, tenofovir, tenofovir disoproxil fumarate, or tenofovir alafenamide. In some embodiments, the NA therapy is entecavir. In some embodiments, the NA therapy is tenofovir. In some embodiments, the NA therapy is tenofovir disoproxil fumarate. In some embodiments, the NA therapy is tenofovir alafenamide.

In another embodiment, the subject is not on NA therapy. In some embodiments, the subject is treatment-naïve.

In some embodiments, the human with a HBsAg baseline level of not greater than a threshold level has a higher chance of achieving HBsAg below LLOQ at the end of the ASO (e.g. bepirovirsen) and interferon treatment as compared to those with a HBsAg baseline level of greater than the threshold level. In some embodiments, the human has at least 5%, 10%, 15%, 20%, 25%, or 30% higher chance of achieving HBsAg below LLOQ at the end of the ASO (e.g. bepirovirsen) and interferon treatment.

In some embodiments, the human with a HBsAg baseline level of not greater than a threshold level has a higher chance of achieving HBV DNA below LLOQ at the end of the ASO (e.g. bepirovirsen) and interferon treatment as compared to those with a HBsAg baseline level of greater than the threshold level. In some embodiments, the human has at least 5%, 10%, 15%, 20%, 25%, or 30% higher chance of achieving HBV DNA below LLOQ at the end of the ASO (e.g. bepirovirsen) and interferon treatment.

In some embodiments, the human with a HBsAg baseline level of not greater than a threshold level has a higher chance of achieving HBsAg and HBV DNA below LLOQ at the end of the ASO (e.g. bepirovirsen) and interferon treatment as compared to those with a HBsAg baseline level of greater than the threshold level. In some embodiments, the human with a HBsAg baseline level of not greater than a threshold level has at least 5%, 10%, 15%, 20%, 25%, or 30% higher chance of achieving HBsAg and HBV DNA below LLOQ at the end of the ASO (e.g. bepirovirsen) and interferon treatment.

In some embodiments, the human with a HBsAg baseline level of not greater than a threshold level has at least 5%, 10%, 15%, 20%, 25%, or 30% higher chance of achieving functional cure as compared to those with a HBsAg baseline level of greater than the threshold level.

In some embodiments, the hepatitis B virus infection is caused by any of the human geographical genotypes, including but not limited to: A (Northwest Europe, North America, Central America); B (Indonesia, China, Vietnam); C (East Asia, Korea, China, Japan, Polynesia, Vietnam); D (Mediterranean area, Middle East, India); E (Africa); F (Native Americans, Polynesia); G (United States, France); or H (Central America).

In some embodiments, the human is HBeAg negative or HBeAg positive prior to treatment. In some embodiments, the human is HBeAg negative prior to treatment. In some embodiments, the human is HBeAg positive prior to treatment.

In some embodiments, the human's HBV DNA and HBsAg levels are below the lower limit of quantification (LLOQ) 24 weeks after stopping all HBV therapy. In some embodiments, the human achieves a functional cure.

In another aspect, the present disclosure provides an ASO (e.g. bepirovirsen) and interferon for use in a method of treating chronic hepatitis B in a human in need thereof, wherein the human has a HBsAg baseline level of not greater than a threshold level.

In another aspect, the present disclosure provides an ASO (e.g. bepirovirsen) and interferon, in particular PegIFN, for use in a method of treating chronic hepatitis B in a human in need thereof, the method comprising:
(a) determining that the human has a HBsAg baseline level of not greater than a threshold level; and
(b) administering to the human a therapeutically effective amount of the ASO (e.g. bepirovirsen) and a therapeutically effective amount of interferon.

### Compositions

an ASO (e.g. bepirovirsen) and interferon may each be incorporated into pharmaceutical compositions for use in the treatment of CHB as described herein. In one embodiment, a pharmaceutical composition comprises an ASO (e.g. bepirovirsen) in combination with one or more pharmaceutically acceptable carriers and/or excipients. In another embodiment, a pharmaceutical composition comprises interferon, in particular PegIFN, in combination with one or more pharmaceutically acceptable carriers and/or excipients.

Such compositions comprise a pharmaceutically acceptable carrier and/or excipient as known and called for by acceptable pharmaceutical practice.

Pharmaceutical compositions may be administered by injection or continuous infusion (examples include, but are not limited to, intravenous, intraperitoneal, intradermal, subcutaneous, intramuscular, intraocular, and intraportal). In one embodiment, the composition is suitable for subcutaneous administration.

In an embodiment, the composition comprising the ASO (e.g. bepirovirsen) also comprises water for injection and has been adjusted to pH 8.0 with acid or base.

In an embodiment, the composition comprising PegIFN also comprises sodium chloride, polysorbate 80, benzyl alcohol, sodium acetate, acetic acid and water for injection. In an embodiment, the PegIFN is PEGASYS. In an embodiment, the PegIFN is PEGINTRON or PEGBERON.

The present disclosure provides a composition comprising an ASO (e.g. bepirovirsen) and a composition comprising interferon for treating a chronic hepatitis B virus infection in a subject in need thereof, the method of treatment comprising administering to the subject a therapeutically effective amount of the ASO (e.g. bepirovirsen) and a therapeutically effective amount of interferon, wherein the subject has a HBsAg baseline level of not greater than a threshold level.

The present disclosure also provides an ASO (e.g. bepirovirsen) and interferon for treating a chronic hepatitis B virus infection in a subject in need thereof, the method comprising administering to the subject a therapeutically effective amount of the ASO (e.g. bepirovirsen) and a therapeutically effective amount of interferon, wherein the subject has a HBsAg baseline level of not greater than a threshold level.

The present disclosure also provides a composition comprising an ASO (e.g. bepirovirsen), or the ASO, and a composition comprising interferon, or interferon, for use in a method of treating chronic hepatitis B in a human in need thereof, wherein the method comprises: (a) determining that the human has a HBsAg baseline level of not greater than a threshold level; and (b) administering to the human a therapeutically effective amount of the ASO (e.g. bepirovirsen) and a therapeutically effective amount of interferon.

In other embodiments, the ASO (e.g. bepirovirsen) composition or the ASO, and the interferon composition or interferon, the method of administering the ASO composition or the ASO, and the interferon composition or interferon, can be according to any of the methods detailed above.

For example, in some embodiments, the threshold level of the HBsAg baseline is about 1000, 1500, 2000, 2500, or 3000 IU/mL, or in a range between any two of the preceding numbers. In some embodiments, the threshold level of the HBsAg baseline is about 1000, 2000, or 3000 IU/mL. In some embodiments, the threshold level of the HBsAg baseline is about 1000 IU/mL. In some embodiments, the threshold level of the HBsAg baseline is about 3000 IU/mL.

In one embodiment, the present disclosure provides an ASO (e.g. bepirovirsen) and interferon for use in a method of treating CHB in a human in need thereof, wherein the method comprises administering to the human a therapeutically effective amount of the ASO (e.g. bepirovirsen), wherein the human has a HBsAg baseline level of not greater than about 1000 IU/mL. In one embodiment, the present disclosure provides an ASO (e.g. bepirovirsen) and interferon for use in a method of treating CHB in a human in need thereof, the method comprising administering to the human a therapeutically effective amount of the ASO (e.g. bepirovirsen) and a therapeutically effective amount of interferon, wherein the human has a HBsAg baseline level of not greater than about 3000 IU/mL.

In another embodiment, the present disclosure provides an ASO (e.g. bepirovirsen) and interferon for use in a method of treating CHB in a human in need thereof, wherein the method comprises administering the ASO (e.g. bepirovirsen) to the human at a dose of 300 mg once weekly for 24 weeks, wherein the human has a HBsAg baseline level of not greater than about 1000 IU/mL. In one embodiment, the present disclosure provides an ASO (e.g. bepirovirsen) and interferon for use in a method of treating CHB in a human in need thereof, the method comprising administering the ASO (e.g. bepirovirsen) to the human at a dose of 300 mg once weekly for 24 weeks, wherein the human has a HBsAg baseline level of not greater than about 3000 IU/mL. In an embodiment, PegIFN alpha-2a is to be administered weekly to the human at a dose of about 180 μg for 24 weeks to 48 weeks following the ASO (e.g. bepirovirsen) cessation. In an embodiment, PegIFN alpha-2a is to be administered weekly to the human at a dose of about 180 μg for 24 weeks following the ASO (e.g. bepirovirsen) cessation. In an embodiment, PegIFN alpha-2a is to be administered weekly to the human at a dose of about 180 μg for 36 weeks following the ASO (e.g. bepirovirsen) cessation. In an embodiment, PegIFN alpha-2a is to be administered weekly to the human at a dose of about 180 μg for 48 weeks following the ASO (e.g. bepirovirsen) cessation.

In another embodiment, the present disclosure provides an ASO (e.g. bepirovirsen) and interferon for use in a method of treating CHB in a human in need thereof, wherein the method comprises administering the ASO (e.g. bepirovirsen) to the human at a dose of 300 mg once weekly for 12 weeks, wherein the human has a HBsAg baseline level of not greater than about 1000 IU/mL. In one embodiment, the present disclosure provides an ASO (e.g. bepirovirsen) and interferon for use in a method of treating CHB in a human in need thereof, the method comprising administering the ASO (e.g. bepirovirsen) to the human at a dose of 300 mg once weekly for 12 weeks, wherein the human has a HBsAg baseline level of not greater than about 3000 IU/mL. In an embodiment, PegIFN alpha-2a is to be administered weekly to the human at a dose of about 180 μg for 24 weeks to 48 weeks following the ASO (e.g. bepirovirsen) cessation. In an embodiment, PegIFN alpha-2a is to be administered weekly to the human at a dose of about 180 μg for 24 weeks following the ASO (e.g. bepirovirsen) cessation. In an embodiment, PegIFN alpha-2a is to be administered weekly to the human at a dose of about 180 μg for 36 weeks following the ASO (e.g. bepirovirsen) cessation. In an embodiment, PegIFN alpha-2a is to be administered weekly to the human at a dose of about 180 μg for 48 weeks following the ASO (e.g. bepirovirsen) cessation.

### Kits

In an aspect of the invention, an ASO (e.g. bepirovirsen) and interferon, in particular PegIFN, are included in a kit with instructions for use. In an embodiment, the kit comprises the ASO (e.g. bepirovirsen) and interferon as disclosed herein, in separate containers.

For convenience, the kit may comprise the ASO (e.g. bepirovirsen) and interferon in predetermined amounts with instructions for use. In an embodiment, the kit comprises 300 mg of the ASO (e.g. bepirovirsen). In an embodiment, the kit comprises 180 μg of PegIFN alpha-2a. In an embodiment, the kit comprises multiples of these unit doses of the ASO (e.g. bepirovirsen) and interferon. In an embodiment, the interferon is interferon alpha or interferon lambda. In another embodiment, the interferon is PegIFN alpha-2a. In an embodiment the kit comprises 1 mL of PegIFN alpha-2a solution for injection in a vial (Type I glass) with stopper (rubber butyl). In an embodiment the kit comprises 1 mL of the ASO (e.g. bepirovirsen) solution for injection in a vial (Type I glass) with stopper (rubber) and capped with an aluminum overseal.

The kit may also include a device to be used for administration of the ASO (e.g. bepirovirsen) and/or a device to be used for administration of interferon, in particular PegIFN alpha-2a. In an embodiment, each unit dose of the ASO (e.g. bepirovirsen) is in a prefilled syringe. In an embodiment, the prefilled syringe is for subcutaneous injection. In an embodiment, each unit dose of interferon is in a prefilled syringe. In an embodiment, the prefilled syringe is for subcutaneous injection.

In a further embodiment, the kit comprises one or more additional medicaments.

### EMBODIMENTS

1. A method for treating chronic hepatitis B in a human in need thereof, the method comprising administering to the human a therapeutically effective amount of bepirovirsen and a therapeutically effective amount of interferon, wherein the human has a HBsAg baseline level of not greater than a threshold level.

2. A method for treating chronic hepatitis B in a human in need thereof, the method comprising:
(a) determining that the human has a HBsAg baseline level of not greater than a threshold level; and
(b) administering to the human a therapeutically effective amount of bepirovirsen and a therapeutically effective amount of interferon.

3. A method for treating chronic hepatitis B in a human in need thereof, the method comprising:
(a) measuring the HBsAg baseline level of the human;
(b) comparing the HBsAg baseline level with a threshold level; and
(c) if the HBsAg baseline level is not greater than the threshold level, then administering to the human a therapeutically effective amount of bepirovirsen and a therapeutically effective amount of interferon.

4. The method of any one of the preceding Embodiments, wherein the threshold level is in the range of about 1000 IU/mL to 3000 IU/mL.

5. The method of any one of the preceding Embodiments, wherein the threshold level is about 1000 IU/mL.

6. The method of any one of Embodiments 1 to 4, wherein the threshold level is about 3000 IU/mL.

7. The method of any one of the preceding Embodiments, wherein the human is not treated with another HBsAg reducing agent or another immunomodulating agent.

8. The method of any one of the preceding Embodiments, wherein bepirovirsen is administered by subcutaneous injection.

9. The method of any one of the preceding Embodiments, wherein bepirovirsen is administered at a dose of about 150 mg or 300 mg once weekly.

10. The method of any one of the preceding Embodiments, wherein bepirovirsen is administered for 12 weeks to 48 weeks.

11. The method of Embodiments 9 or 10, wherein bepirovirsen is administered weekly with additional loading doses in the first two weeks on Day 4 and Day 11 following the first dose.

12. The method of any one of the preceding Embodiments, wherein bepirovirsen is administered at a dose of 300 mg once weekly for 12 weeks.

13. The method of any one of Embodiments 1 to 11, wherein bepirovirsen is administered at a dose of 300 mg once weekly for 24 weeks.

14. The method of any one of the preceding Embodiments, wherein interferon is interferon alpha.

15. The method of any one of Embodiments 1 to 13, wherein interferon is interferon lambda.

16. The method of Embodiment 14, wherein interferon alpha is pegylated human interferon alpha-2a.

17. The method of Embodiment 16, wherein PegIFN is administered at a dose of about 180 μg once weekly.

18. The method of any one of the preceding Embodiments, wherein interferon is administered by subcutaneous injection.

19. The method of any one of the preceding Embodiments, wherein interferon is administered following cessation of bepirovirsen.

20. The method of any one of the preceding Embodiments, wherein interferon is administered for 12 weeks to 24 weeks following cessation of bepirovirsen.

21. The method of any one of the preceding Embodiments, wherein the human is on stable nucleoside or nucleotide analogue (NA) therapy.

22. The method of Embodiment 21, wherein the NA therapy is selected from the group consisting of: lamivudine, adefovir, adefovir dipivoxil, telbivudine, entecavir, tenofovir, tenofovir disoproxil fumarate, tenofovir alafenamide, and a pharmaceutically acceptable salt of any of the foregoing.

23. The method of Embodiment 21 or Embodiment 22, wherein the human discontinues NA therapy 24 weeks after interferon cessation.

24. The method of any one of Embodiments 1 to 20, wherein the human is not treated with a nucleoside or nucleotide analogue (NA).

25. The method of any one of the preceding Embodiments, wherein the human does not have liver cirrhosis.

26. The method of any one of the preceding Embodiments, wherein the human is HBeAg negative prior to the bepirovirsen treatment.

27. The method of any one of Embodiments 1 to 25, wherein the human is HBeAg positive prior to the bepirovirsen treatment.

28. The method of any one of the preceding Embodiments, wherein the human's HBV DNA and HBsAg levels are below the lower limit of quantification (LLOQ) 24 weeks after stopping all HBV therapy.

29. A combination of bepirovirsen and PegIFN alpha-2a for the treatment of chronic hepatitis B in a human, wherein:
(a) the human to be treated has a baseline HBsAg≤3000 IU/mL;
(b) bepirovirsen is to be administered to the human weekly at a dose of about 300 mg for 12 weeks or 24 weeks; and
(c) PegIFN alpha-2a is to be administered to the human weekly at a dose of about 180 μg for 12 weeks to 24 weeks following cessation of bepirovirsen.

30. A combination of bepirovirsen and PegIFN alpha-2a for achieving a functional HBV cure in a human with chronic hepatitis B, wherein:
(a) the human to be treated has a baseline HBsAg≤3000 IU/mL;
(b) bepirovirsen is to be administered to the human weekly at a dose of about 300 mg for 12 weeks or 24 weeks; and
(c) PegIFN alpha-2a is to be administered to the human weekly at a dose of about 180 μg for 12 weeks to 24 weeks following cessation of bepirovirsen.

31. Use of bepirovirsen in the manufacture of a medicament for the treatment of chronic hepatitis B in a human, wherein:
(a) the human to be treated has a baseline HBsAg≤3000 IU/mL;
(b) bepirovirsen is to be administered to the human weekly at a dose of about 300 mg for 12 weeks or 24 weeks; and
(c) PegIFN alpha-2a is to be administered weekly to the human at a dose of about 180 μg for 12 weeks to 24 weeks following cessation of bepirovirsen.

32. Use of PegIFN alpha-2a in the manufacture of a medicament for the treatment of chronic hepatitis B in a human, wherein:
(a) the human to be treated has a baseline HBsAg≤3000 IU/mL;
(b) bepirovirsen is to be administered to the human weekly at a dose of about 300 mg for 12 weeks or 24 weeks; and
(c) PegIFN alpha-2a is to be administered to the human weekly at a dose of about 180 μg for 12 weeks to 24 weeks following cessation of bepirovirsen.

### EXAMPLES

While aspects of the disclosure presented herein have been described more particularly in accordance with some embodiments, the following examples, which highlight certain features and properties of the exemplary embodiments of the disclosure described herein, serve only to illustrate the disclosure described herein and are not intended to limit the same.

### Example 1

Phase IIb Study of Sequential Bepirovirsen and Peginterferon (PegIFN) Treatment in Participants with Chronic Hepatitis B (B-Together Study)

### A. Study Design

This is a Phase IIb, multi-centre, randomised, open-label study to assess the efficacy and safety of sequential treatment with bepirovirsen (300 mg/week for 12 or 24 weeks) followed by PegIFN (PEGASYS, 180 mcg/week for up to 24 weeks) in participants with chronic hepatitis B on stable NA therapy (B-Together). During the bepirovirsen treatment period, a 300 mg loading dose will be administered on Day 4 and Day 11. Participants will continue their NA therapy for the duration of the study.

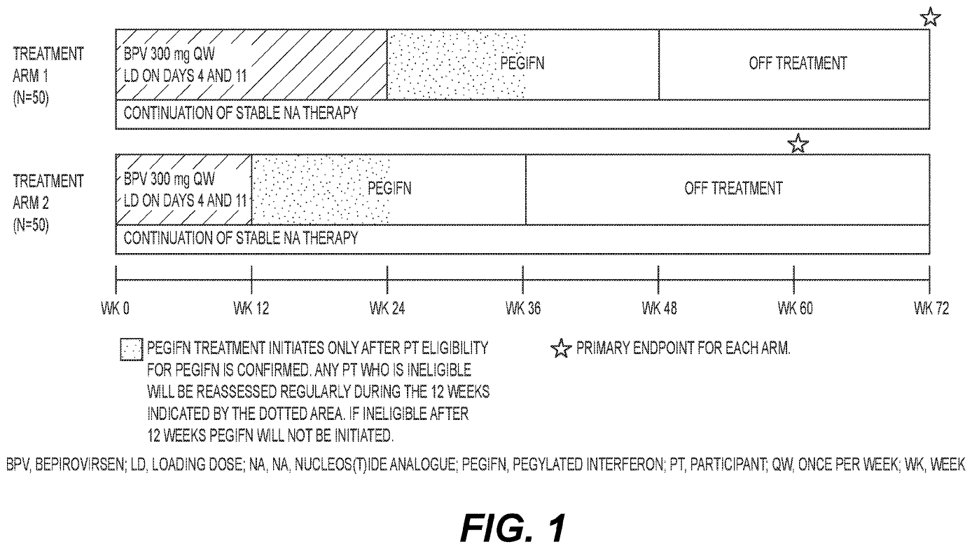

Participants will be randomised (1:1) into one of two parallel treatment arms (FIG. 1).
1. 300 mg bepirovirsen once/week for 24 weeks (plus loading dose on Day 4 and 11) followed by PegIFN (180 mcg/week for up to 24 weeks). Participants will be followed up for an additional 24 weeks (off-treatment phase) once PegIFN treatment is complete.
2. 300 mg bepirovirsen once/week for 12 weeks (plus loading dose on Day 4 and 11) followed by PegIFN (180 mcg/week for up to 24 weeks). Participants will be followed up for an additional 36 weeks (off-treatment phase) once PegIFN treatment is complete.

The planned duration of bepirovirsen is 24 weeks and 12 weeks in Treatment Arm 1 and 2 respectively. At the end of the planned bepirovirsen dosing period, participants will be reassessed for eligibility to start PegIFN treatment based on the label.
1. Participants immediately eligible for PegIFN will begin treatment.
2. Participants who are not considered eligible will be re-assessed for eligibility and may delay start of PegIFN treatment for up to 12 weeks.
3. Participants who fail to meet PegIFN eligibility criteria by PegIFN Eligibility Week 13 (i.e., can no longer receive at least 12 weeks of PegIFN treatment) will not receive treatment with PegIFN.
   Note: all participants are expected to end PegIFN treatment at the same time, regardless of when they start therapy (Treatment Group 1: all participants should complete treatment by Week 48; Treatment Group 2: Week 36). As such, the planned treatment duration with PegIFN may vary from a minimum of 12 weeks to a maximum of 24 weeks.

Populations will be stratified based on HBsAg level (HBsAg≤3 log IU/mL and >3 log IU/mL) and whether participants are HBeAg positive or negative.

In the B-Clear study to evaluate efficacy of bepirovirsen monotherapy, the primary analysis was conducted separately for each of the 2 cohorts (participants stably maintained on nucleos (t) ide analogues (NA) background therapy [On-NA] or not currently on NA therapy [Not-on-NA]). See Yuen et al., N. Engl. J. Med. 2022; 387:1957-68. Participants were randomly assigned (in a 3:3:3:1 ratio) to receive weekly subcutaneous injections of bepirovirsen at a dose of 300 mg for 24 weeks (group 1), bepirovirsen at a dose of 300 mg for 12 weeks then 150 mg for 12 weeks (group 2), bepirovirsen at a dose of 300 mg for 12 weeks then placebo for 12 weeks (group 3), or placebo for 12 weeks then bepirovirsen at a dose of 300 mg for 12 weeks (group 4). The primary composite efficacy outcome was an HBsAg level below the lower limit of detection (0.05 IU/mL) and an HBV DNA level below the lower limit of quantification (LLOQ) (20 IU/mL) maintained for 24 weeks after the planned end of bepirovirsen treatment in the absence of any medication initiated for the purpose of suppressing HBV replication (groups 1, 2, and 3)

In group 1, a primary-outcome event occurred in 6 participants (9%) receiving NA therapy and in 7 (10%) not receiving NA therapy. In group 2, a primary-outcome event occurred in 6 participants (9%) receiving NA therapy and in 4 (6%) not receiving NA therapy. In group 1, a total of 16% of the participants receiving NA therapy and 25% of those not receiving NA therapy with a low HBsAg level (≤3 log 10 IU per milliliter) at baseline had a primary-outcome event, as compared with 6% and 7% of participants, respectively, with a high HBsAg level (>3 log 10 IU per milliliter) at baseline.

The B-Together study was designed to assess if sequential bepirovirsen and PegIFN dual therapy could improve the efficacy rates observed for bepirovirsen monotherapy in the B-Clear study, in particular whether up to 24 weeks of treatment with bepirovirsen followed by up to 24 weeks of PegIFN can increase the rate of hepatitis B virus surface antigen (HBsAg) loss in participants on stable nucleos (t) ide analogue (NA) therapy, and whether virologic response can be sustained once PegIFN treatment is discontinued.

The study was designed to answer several questions and used an innovative approach to address design challenges: (1) when it is optimal to add PegIFN; (2) whether the sequential addition of PegIFN can convert bepirovirsen partial HBsAg responder to full responder; and (3) whether the addition of PegIFN can improve durability of response off-treatment. The sequential design and eligibility period between bepirovirsen and PegIFN treatment were implemented to manage the potential overlapping toxicity risk (such as platelet decline). Frequent PegIFN eligibility assessment visits after the end of bepirovirsen treatment ensure adequate safety monitoring and timely initiation of PegIFN treatment. At the time of study design, there was limited bepirovirsen safety data beyond 4 weeks duration. To demonstrate therapeutic effect, the design relied heavily on indirect comparisons using historical data for PegIFN and available bepirovirsen monotherapy data.

### B. Objectives and Estimands

The primary, secondary and exploratory objectives and estimands and endpoints are:

| | |
|---|---|
| Objectives | Estimands and Endpoints |

Primary

| | |
|---|---|
| Efficacy: To investigate the efficacy | The main Estimand supporting the primary objective |
| of two different durations of | is defined as: |
| bepirovirsen followed by up to 24 | Population: Participants with CHB on stable NA |
| weeks of PegIFN therapy in | therapy |
| participants with CHB on stable | Treatment: 300 mg bepirovirsen for 12 or 24 |
| NA therapy. | weeks followed by up to 24 weeks of PegIFN |
| | therapy while on stable NA therapy |
| | Variable (Categorical): Participants achieving |
| | Sustained Virologic Response (SVR) (HBsAg <LLOQ |
| | and HBV DNA <LLOQ) for 24 weeks after the |
| | planned end of sequential treatment, |
| | without use of any rescue medication |
| | Intercurrent Events: |
| | Discontinuation of, interruption in, and |
| | nonadherence to bepirovirsen and PegIFN |
| | not related to any wide disruptive events |
| | (such as COVID-19 pandemic) will be |
| | ignored (treatment policy strategy) |
| | Ineligibility to receive PegIFN will be |
| | ignored (treatment policy strategy) |
| | Use of rescue medication (composite strategy) |
| | Wide disruptive events (such as COVID-19 |
| | pandemic) leading to discontinuation of, |
| | interruption in, and non-adherence to |
| | bepirovirsen and PegIFN will be handled |
| | assuming they had not happened |
| | (hypothetical strategy). |
| | Population Summary: The percentage of |
| | participants in each treatment group who achieve |
| | SVR, without use of any rescue medication |
| | The main primary estimand supporting the primary |
| | objective in participants with CHB on stable NA |
| | therapy in each treatment arm is the percentage of |
| | participants that achieve SVR (HBsAg <LLOQ and |
| | HBV DNA <LLOQ) for 24 weeks after the planned |
| | end of sequential |
| | treatment in the absence of rescue medication, |
| | regardless of ineligibility to receive PegIFN, |
| | discontinuation of, interruption in, and non- |
| | adherence to bepirovirsen and PegIFN, had they not |
| | been affected by wide disruptive events. |
| | Three supplementary Estimands are defined to |
| | support the primary objective: |
| | The first supplementary Estimand is defined in |
| | the same way as the main Estimand, except the |
| | assessment time frame for patients achieving |
| | SVR will be 24 weeks after the actual end of |
| | treatment. Therefore, the strategy for |
| | intercurrent events of treatment discontinuation |
| | will be while-on-treatment. This supplementary |
| | estimand supporting the primary objective in |
| | participants with CHB on stable NA therapy in |
| | each treatment arm is the percentage of |
| | participants that achieve SVR (HBsAg <LLOQ |
| | and HBV DNA <LLOQ) for 24 weeks after the |
| | actual end of sequential treatment in the |
| | absence of rescue medication, regardless of |
| | ineligibility to receive PegIFN, discontinuation |
| | of, interruption in, and nonadherence to |
| | bepirovirsen and PegIFN, had they not been |
| | affected by wide disruptive events. |
| | The second supplementary Estimand is to |
| | understand the relationship between the PegIFN |
| | duration and achieving SVR for 24 weeks after |
| | the actual end of treatment, defined as: |
| | Population: Participants with CHB on |
| | stable NA therapy |
| | Treatment: 300 mg bepirovirsen for 12 or |
| | 24 weeks followed by 24 weeks of PegIFN |
| | therapy while on stable NA therapy |
| | Variable: The relationship between SVR |
| | for 24 weeks after actual end of treatment |
| | and the duration of PegIFN received by |
| | participants |
| | Intercurrent Events: |
| | Discontinuation and delayed start of, |
| | PegIFN, will be accounted to reflect |
| | the actual duration from the first to the |
| | last dose of PegIFN received (while- |
| | on-treatment strategy). |
| | Interruption in and other non- |
| | adherence to PegIFN will be ignored |
| | (treatment policy strategy) |
| | Discontinuation of, interruption in and |
| | non-adherence to bepirovirsen will be |
| | ignored (treatment policy strategy) |
| | Use of rescue medication (composite |
| | strategy). |
| | Wide disruptive events (such as |
| | COVID-19 pandemic) leading to |
| | discontinuation and delayed start of, |
| | PegIFN will be handled with while-on- |
| | treatment strategy; wide disruptive |
| | events leading to interruption in and |
| | other non-adherence to PegIFN will be |
| | ignored (treatment policy strategy); |
| | wide disruptive events leading to |
| | discontinuation of, interruption in, and |
| | non-adherence to bepirovirsen will be |
| | ignored (treatment policy strategy). |
| | Population Summary: The percentage of |
| | participants achieving SVR for 24 weeks |
| | after the actual end of treatment by |
| | PegIFN treatment duration categorical |
| | grouping in each treatment arm |
| | The second supplementary Estimand |
| | (supporting the primary objective in participants |
| | with CHB on stable NA therapy) is the |
| | percentage of participants achieving SVR for 24 |
| | weeks after the actual end of treatment by |
| | PegIFN treatment duration categorical grouping |
| | in each treatment arm, taking into account |
| | discontinuation and delayed start of PegIFN, |
| | regardless of interruption in and other |
| | nonadherence to PegIFN, regardless of |
| | discontinuation of, interruption in and non- |
| | adherence to bepirovirsen. |
| | The third supplementary Estimand is defined in |
| | the same way as the main Estimand, except the |
| | strategy for intercurrent events of PegIFN |
| | ineligibility for more than 12 weeks and/or |
| | missing more than 12 doses of PegIFN will be |
| | principal stratum. This supplementary Estimand |
| | supporting the primary objective is the |
| | percentage of participants in each treatment arm |
| | that achieve SVR (HBsAg <LLOQ and HBV |
| | DNA <LLOQ) for 24 weeks after the planned |
| | end of sequential treatment in participants with |
| | CHB on stable NA therapy and receiving at least |
| | 12 doses of PegIFN, in the absence of rescue |
| | medication, regardless of discontinuation of, |
| | interruptions in or non-adherence to bepirovirsen |
| | had they not been affected by wide disruptive |
| | events. |

Secondary

| | |
|---|---|
| Efficacy: To assess the efficacy of | The Estimand supporting the objective is defined as: |
| bepirovirsen and PegIFN sequential | Population: Participants with CHB on stable |
| therapy on biomarkers and virus | NA therapy; For the time to ALT normalisation |
| specific antibody responses | variable, population will be aforementioned |
| | participants with baseline ALT > ULN. |
| | Treatment: 300 mg bepirovirsen for 12, or 24 |
| | weeks followed by 24 weeks of PegIFN therapy |
| | while on stable NA therapy |
| | Categorical Variables: |
| | Achieving HBsAg <LLOQ and HBV DNA <LLOQ |
| | at two time points: (1) the planned |
| | end of treatment and (2) at the end of 24 |
| | weeks follow-up |
| | Categorical changes from baseline in |
| | HBsAg (e.g., <0.5, ≥0.5, ≥1, ≥1.5, ≥3 log10 |
| | IU/mL) at each scheduled visit or analysis |
| | window. |
| | ALT normalisation (ALT ≤ ULN) over time |
| | in absence of rescue medication in |
| | participants with baseline ALT > ULN |
| | Population summary is the percentage of |
| | participants in each category for each |
| | treatment group. |
| | HBe antibody (anti-HBeAg) levels. |
| | Continuous Variables: |
| | Actual values and change from baseline |
| | over time for HBsAg, HBV DNA and HBeAg |
| | Actual values and change from baseline |
| | over time for HBs antibody (anti-HBsAg) |
| | levels over time |
| | Actual values and change from baseline |
| | over time for ALT |
| | Population summary is the mean values and |
| | the mean changes from baseline of each |
| | variable for participants in each treatment |
| | group. |
| | Time to Event Variable: |
| | Time to ALT normalisation in absence of |
| | rescue medication in participants with |
| | baseline ALT > ULN |
| | Population summary is the Turnbull's |
| | estimator for non-parametric estimation of |
| | Time to ALT normalisation in each treatment arm |
| | Intercurrent Events: Discontinuation of, |
| | interruption in, and non-adherence to |
| | bepirovirsen and PegIFN will be ignored |
| | (treatment policy); PegIFN ineligibility will be |
| | ignored (treatment policy); Rescue medication |
| | will be ignored (treatment policy), except for |
| | ALT normalisation which can only be achieved |
| | in the absence of rescue medication; Wide |
| | disruptive events (such as COVID-19 pandemic) |
| | leading to discontinuation of, interruption in, and |
| | nonadherence to bepirovirsen and PegIFN will be |
| | handled with Treatment Policy Strategy. |
| | The group of estimands supporting this objective |
| | is the population summary for each variable in |
| | each treatment arm in the population regardless |
| | of discontinuation of, interruption in or non- |
| | adherence to bepirovirsen and PegIFN, and |
| | regardless of rescue medication (except for ALT |
| | normalization which can only be achieved in the |
| | absence of rescue medication) or PegIFN |
| | ineligibility. |
| Efficacy: To investigate the durability | The same definition as the above secondary |
| of virological response after | estimand, focusing on the timepoints following the |
| sequential therapy with 12 weeks of | 24 weeks offtreatment period in the treatment arm of |
| bepirovirsen followed by 24 weeks of | 300 mg bepirovirsen for 12 weeks followed by up to |
| PegIFN in participants with CHB on | 24 weeks of PegIFN therapy while on stable NA |
| stable NA therapy for up to 36 weeks | therapy. |
| off treatment. | |
| Efficacy: To compare efficacy between | The same definition as the primary estimand except |
| different treatment durations: | treatments and population summary are defined as: |
| 12 or 24 weeks of bepirovirsen | Treatments: Arms 1 and 2. One treatment |
| followed by 24 weeks PegIFN | comparison between Arms 1 & 2 up to 24 |
| | weeks off-treatment |
| | Population summary: difference in proportion of |
| | participants who achieve SVR between treatment arms |
| | The group of estimands supporting this objective in |
| | participants with CHB on stable NA therapy is the |
| | difference between treatment arms 1 and 2 in the |
| | proportion of participants that achieve SVR for 24 |
| | weeks |
| | after the planned end of sequential treatment, in the |
| | absence of rescue medication, regardless of |
| | ineligibility to receive PegIFN, regardless of |
| | discontinuation of, interruption in or non-adherence |
| | to bepirovirsen and PegIFN had they not been |
| | affected by wide disruptive events. |

Exploratory

| | |
|---|---|
| Pharmacokinetics (PK) and PK- | Efficacy assessments for PK/PD relationship include |
| pharmacodynamic (PD) | but not limited to |
| relationships of bepirovirsen and | Categorical: virologic response, HBsAg level <LLOQ, |
| PegIFN: To evaluate PK, PK-efficacy | HBV DNA level <LLOQ, and seroconversion |
| relationship and PK-safety relationship | (anti-HBsAg and anti-HBeAg); |
| | Change from baseline: HBsAg, HBV DNA, |
| | anti-HBsAg and anti-HBeAg levels; |
| | Time to event: virologic response (HBsAg and |
| | HBV DNA levels <LLOQ), nadir of HBsAg |
| | and HBV DNA, HBsAg level <LLOQ, HBV |
| | DNA level <LLOQ, seroconversion (anti-HBsAg |
| | and anti-HBeAg), and peak of ALT flares |
| | Safety assessments for PK/PD relationship include |
| | but not limited to vital signs, laboratory |
| | measurements and adverse events |
| | PK assessments include but not limited to Cτ and |
| | terminal half-life (t½). |
| Safety and Tolerability: To | Clinical assessments including laboratory |
| investigate the safety profile of | measurements and adverse events at specified |
| sequential therapy with | timepoints |
| bepirovirsen (up to 24 weeks) | |
| followed by PegIFN (up to 24 weeks) | |
| in participants with CHB on stable | |
| NA therapy. | |
| Efficacy: To assess the pharmacodynamic | HBV core related antigen (HBcrAg), HBV RNA at |
| effect of bepirovirsen and PegIFN | specified timepoints |
| sequential therapy on exploratory | |
| viral biomarkers | |
| Virology: To assess the effect of | Sequencing of the viral HBV DNA and/or HBV |
| genotype and presence of baseline | RNA prior to treatment, during treatment and post |
| polymorphisms within the bepirovirsen | treatment visits at specified timepoints |
| binding site. | |
| The effect of genotype and | |
| polymorphisms on treatment response | |
| will be assessed. | |
| To assess the emergence of mutations | |
| within the GSK binding site, and | |
| elsewhere in the hepatitis B genome, | |
| during and after treatment will be | |
| assessed | |
| Biomarkers: To assess the effect of | Laboratory measurements of and correlation |
| bepirovirsen and PegIFN sequential | between the following at specified timepoints: |
| therapy on immunological biomarkers. | Virological biomarkers, including specific |
| To describe the relationship(s) | viral parameters (HBsAg, HBeAg, HBV |
| between virology biomarkers including | DNA, HBV RNA, HBcrAg) |
| HBsAg and immunological biomarkers. | Soluble biomarkers, including levels of |
| | circulating cytokines and chemokines |
| | Markers of immune cell function, including |
| | relative frequencies of immune cell subsets |
| | among PBMCs, activation status as |
| | determined by phenotyping and gene expression |
| | patterns, and functional assays including HBV- |
| | specific cytokine and/or antibody production |

### C. Study Population: Inclusions and Exclusion Criteria

### Inclusion Criteria Include:

### Age

1. At least 18 to 75 years of age at the time of signing the informed consent [if country/site age requirements for consent differ, the more stringent (e.g., higher age) restriction will be required for that country/site].

### Type of Participant and Disease Characteristics

2. Participants who are eligible to be treated with PegIFN
3. Documented chronic HBV infection ≥6 months prior to screening AND currently receiving stable NA therapy except telbivudine, defined as no changes to their NA regimen from at least 6 months prior to screening and with no planned changes to the stable regimen over the duration of the study
4. Plasma or serum HBsAg concentration >100 IU/mL
5. Plasma or serum HBV DNA<90 IU/mL
6. Alanine Transaminase (ALT)≤2×ULN

### Exclusion Criteria Include:

### Medical Conditions

1. Clinically significant abnormalities, aside from chronic HBV infection in medical history (e.g., moderate-severe liver disease other than chronic HBV, acute coronary syndrome within 6 months of screening, major surgery within 3 months of screening, significant/unstable cardiac disease, uncontrolled diabetes, bleeding diathesis, autoimmune disease, or coagulopathy) or physical examination
2. Co-infection with:
   a. Current or past history of Hepatitis C virus (HCV)
   b. Human immunodeficiency virus (HIV)
   c. Hepatitis D virus (HDV)
3. History of or suspected liver cirrhosis and/or evidence of cirrhosis as determined by
   a. Both Aspartate aminotransferase (AST)-Platelet Index (APRI)>2 and FibroSure/Fibro Test result >0.7
      i. If only one parameter (APRI or FibroSure/FibroTest) result is positive, a discussion with the Medical Monitor is required before inclusion in study is permitted
   b. Regardless of APRI or Fibrosure/FibroTest score, if the participant meets one of the following historical criteria, they will be excluded from the study
      i. Liver biopsy (i.e., Metavir Score F4)
      ii. Liver stiffness >12 kPa
4. Diagnosed or suspected hepatocellular carcinoma as evidenced by the following
   a. Alpha-fetoprotein concentration ≥200 ng/ml
   b. If the screening alpha fetoprotein concentration is ≥50 ng/ml and <200 ng/ml, the absence of liver mass must be documented by imaging within 6 months before randomisation
5. History of malignancy within the past 5 years with the exception of specific cancers that are cured by surgical resection (e.g., skin cancer), participants under evaluation for possible malignancy are not eligible.
6. History of vasculitis or presence of symptoms and signs of potential vasculitis [e.g., vasculitic rash, skin ulceration, repeated blood detected in urine without identified cause] or history/presence of other diseases that may be associated with vasculitis condition (e.g., systemic lupus erythematosus, rheumatoid arthritis, relapsing polychondritis, mononeuritis multiplex)]
7. History of extrahepatic disorders possibly related to HBV immune conditions (e.g., nephrotic syndrome, any type of glomerulonephritis, polyarteritis nodosa, cryoglobulinemia, uncontrolled hypertension)
8. Poorly controlled thyroid dysfunction or abnormal thyroid stimulating hormone (TSH) levels
9. Positive (or borderline positive) Anti-neutrophil cytoplasmic antibody (ANCA) at screening:
   a. Participants that meet these criteria may be considered for inclusion in the study following:
      i. Analysis of MPO-ANCA [perinuclear anti-neutrophil cytoplasmic antibodies (pANCA)] and PR3-ANCA [classical anti-neutrophil cytoplasmic antibodies (cANCA)] AND
      ii. A discussion with the Medical Monitor to review participant's complete medical history to ensure no past history or current manifestations of a vasculitic/inflammatory/auto-immune condition
10. Low C3 at screening AND evidence of past history or current manifestations of vasculitic/inflammatory/auto-immune conditions
   a. All participants with low C3 at screening should have their medical history discussed with the Medical Monitor prior to enrolment
11. History of alcohol or drug abuse/dependence
   a. Current alcohol use as judged by investigator to potentially interfere with participant compliance
   b. History of or current drug abuse/dependence as judged by the investigator to potentially interfere with participant compliance
      i. Refers to illicit drugs and substances with abuse potential. Medications that are used by the participant as directed, whether over-the-counter or through prescription, are acceptable and would not meet the exclusion criteria
12. Pre-existing severe psychiatric condition or a history of severe psychiatric disorders, including severe depression, suicidal ideation and attempted suicide.

### Prior/Concomitant Therapy

13. Currently taking, or took within 3 months of screening, any immunosuppressing drugs (e.g., prednisone), other than a short course of therapy (≤2 weeks) or topical/inhaled steroid use.
14. Participants for whom immunosuppressive treatment is not advised, including therapeutic doses of steroids.
15. Patients with prior treatment with Pegylated interferon or interferon are excluded.
16. Participants requiring anti-coagulation therapies (for example warfarin, Factor Xa inhibitors or anti-platelet agents like clopidogrel)
17. Participants currently taking, or took within 6 months of screening, telbivudine

### Prior/Concurrent Clinical Study Experience

18. The participant has participated in a clinical trial and has received an investigational product within the following time period prior to the first dosing day in the current study: 5 half-lives (if known) or twice the duration (if known) of the biological effect of the study treatment (whichever is longer) or 90 days (if half-life or duration is unknown).
19. Prior treatment with any oligonucleotide or small interfering RNA (siRNA) within 12 months prior to the first dosing day

### Diagnostic Assessments

20. Fridericia's QT correction formula (QTcF)≥450 msec (if single ECG at screening shows QTcF≥450 msec, a mean of triplicate measurements should be used to confirm that participant meets exclusion criterion).
21. Laboratory results as follows
   a. Serum albumin <3.5 g/dL
   b. Glomerular filtration rate (GFR)<60 mL/min/1.73 m2 as calculated by the Chronic Kidney Disease Epidemiologic Collaboration formula (for Japan, Japanese Society of Nephrology-Chronic Kidney Disease Initiative equation).
   c. International normalised ratio (INR)>1.25
   d. Platelet count <140×109/L
   e. Absolute neutrophil count (ANC)<1.5×109 cells/L
   f. Baseline haemoglobin <10 g/dL
   g. Total bilirubin >1.25×ULN. For participants with benign unconjugated hyperbilirubinemia with total bilirubin >1.25×ULN, discussion for inclusion to the study is required with the Medical Monitor
   h. Urine albumin to creatinine ratio (ACR)≥0.03 mg/mg (or ≥30 mg/g). In the event of an ACR above this threshold, eligibility may be confirmed by a second measurement. In cases where participants have low urine albumin and low urine creatinine levels resulting in a urine ACR calculation ≥0.03 mg/mg (or ≥30 mg/g), the investigator should confirm that participant does not have a history of diabetes, hypertension or other risk factors that may affect renal function and discuss with the medical monitor, or designee.

### Other Exclusions

22. History of/sensitivity to bepirovirsen or interferon-containing therapies, or components thereof, or a history of drug or other allergy that, in the opinion of the investigator or medical monitor, contraindicates their participation

### D. Study Assessment and Procedures

The primary objective measurement for efficacy in this study is the assessment of bepirovirsen treatment followed by PegIFN on serum HBsAg and HBV DNA levels in participants with CHB. The primary efficacy endpoint is achieving serum HBsAg and HBV DNA levels <LLOQ.

Any HBsAg greater than LLOQ after achieving HBsAg seroclearance needs to be confirmed by re-test within 1 week of receiving the test result. The re-test result will be used if the first test is not confirmed.

There are planned time points for all safety assessments. Additional time points for safety tests (such as vital signs, physical exams and laboratory safety tests) may be added during the course of the study based on newly available data to ensure appropriate safety monitoring.

### E. Interim Results

A protocol-specified interim analysis was implemented, once all participants had completed the on-treatment phase of the study. See Table 1.

TABLE 1
Virologic Response in B-Together (Interim Analysis) (Intent-to-Treat Population)

| | Arm 1 | | | Arm 2 | | |
|---|---|---|---|---|---|---|
| | Bepirovirsen (300 mg + LD) × 24 | | | Bepirovirsen (300 mg + LD) × 12 | | |
| | wk + PegIFN × 24 wk (n = 55) | | | wk + PegIFN × 24 wk (n = 53) | | |
| | | HBsAg ≤3000 | HBsAg ≤1000 | | HBsAg ≤3000 | HBsAg ≤1000 |
| | All | IU/mL | IU/mL | All | IU/mL | IU/mL |
| | (n = 55) | (n = 37) | (n = 17) | (n = 53) | (n = 31) | (n = 17) |
| Bepirovirsen EOT VR | 19 (35) | 19 (51) | 11 (65) | 13 (25) | 11 (35) | 11 (65) |
| PegIFN EOT VR | 13 (24) | 13 (35) | 9 (53) | 9 (17) | 9 (29) | 8 (47) |
| VR maintained off- | 8 (15)^{a} | 8 (22)^{a} | 5 (29)^{a} | — | — | — |
| treatment | | | | | | |
| Primary endpoint | 5 (9)^{b} | 5 (14)^{b} | 4 (24)^{b} | 8 (15) | 8 (26) | 7 (41) |

All entries are No. (%) of participants;
VR = Virological Response;
EOT = End of Treatment
Primary efficacy endpoint was assessed using the modified definition: an HBsAg or HBV DNA value ≥LLOQ is considered as virologic failure only if confirmed at the next visit.
^{a}As of the interim analysis participants in this row have not yet reached 24 weeks after the end of sequential treatment and are therefore not yet evaluable for the primary endpoint.
^{b}As of the interim analysis not all participants have had the opportunity to reach the primary endpoint in Arm
1. Therefore, primary endpoint counts and percentages in Arm 1 are preliminary and may change between the interim analysis and End of Study (EOS).

This analysis reported that 8 of 53 (15%) of participants in the 12-week bepirovirsen+24-week PegIFN treatment arm achieved the primary efficacy endpoint of HBsAg and HBV DNA <LLOQ sustained for 24 weeks after completion of PegIFN treatment. The response rate was 26% (8/31) and 41% (7/17) in participants with baseline HBsAg≤3000 and ≤1000 IU/mL, respectively. The point estimates for the primary efficacy endpoint rates observed in B-Together are numerically higher than those observed with 12-week bepirovirsen-only therapy in B-Clear which showed rates of 7% (3/41) and 11% (2/19) in the ≤3000 and ≤1000 IU/mL populations respectively, in participants on stable NA therapy.

As of the interim analysis, data from a second treatment arm exploring 24 weeks of bepirovirsen followed by 24 weeks PegIFN was incomplete; however, this predicts a similar end of study primary efficacy rate in a further 55 participants.

Relapse rates compared between B-Together interim analysis and B-Clear final data show that in B-Clear Arms 1 and 3, the overall relapse rates were 63% and 75%, respectively. By contrast, in B-Together, 4 participants (31%) who achieved virologic response at the end of the bepirovirsen treatment period relapsed by the end of the PegIFN phase. Further, as of the interim analysis, no participants showing primary efficacy response at the end of PegIFN treatment have yet demonstrated relapse.

The additional benefit of 24 weeks PEGASYS in this regimen appears to come from a marked reduction in relapse rate in the 6 months following the end of bepirovirsen treatment, supporting the hypothesis that addition of an immunomodulatory therapy following suppression of viral antigens with bepirovirsen treatment increases the chance of a durable response and the opportunity to achieve functional cure once all therapy is stopped.

### F. End of Study Results

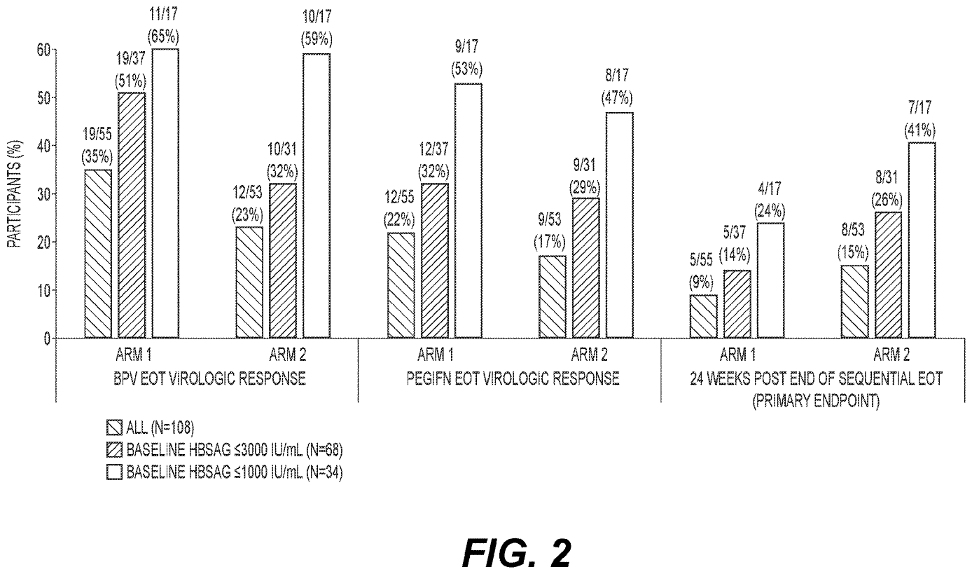

The virologic response data analyzed at the end of study are shown in Table 2. One HBeAg (+) participant on Arm 1 achieved Primary Endpoint (PE) (HBsAg≤1000 IU/mL), and no HBeAg (+) participants on Arm 2 achieved PE. Two participants on Arm 1 maintained response from Peg EOT through majority of Off-Treatment (OT), but had slight HBsAg elevations at OT-W24 only. In both arms, response increased in participants with lower baseline HBsAg levels. See FIG. 2. All responders had baseline HBsAg≤3000 IU/mL. Only Bepirovirsen (BPV) responders benefitted from Peg-IFN treatment, through prevention of relapse off-treatment (relapse rate B-Together vs B-Clear: Arm 1, 58% vs 63%; Arm 2, 0% vs 75%). A higher proportion (30 of 80; 37.5%) of HBeAg negative participants had low HBsAg (≤1000 IU/mL) at baseline compared to HBeAg positive participants (4 of 28, 14.3%)

TABLE 2
Summary of Virologic Response at End of Bepirovirsen, PegIFN, and PE by Baseline HBsAg Categories

| | Arm 1 | | | | | | Arm 2 | | | | | |
|---|---|---|---|---|---|---|---|---|---|---|---|---|
| | Bepirovirsen (300 mg + LD) × 24 | | | | | | Bepirovirsen (300 mg + LD) × 12 | | | | | |
| | wk + PegIFN × 24 wk (n = 55) | | | | | | wk + PegIFN × 24 wk (n = 53) | | | | | |
| | | | HBsAg ≤3000 | | HBsAg ≤1000 | | | | HBsAg ≤3000 | | HBsAg ≤1000 | |
| | All | | IU/mL | | IU/mL | | All | | IU/mL | | IU/mL | |
| | (n = 55) | | (n = 37) | | (n = 17) | | (n = 53) | | (n = 31) | | (n = 17) | |
| Bepirovirsen EOT VR | 19 | (35%) | 19 | (51%) | 11 | (65%) | 12 | (23%) | 10 | (32%) | 10 | (59%) |
| PegIFN EOT VR | 12 | (22%) | 12 | (32%) | 9 | (53%) | 9 | (17%) | 9 | (29%) | 8 | (47%) |
| Relapse off-treatment | 7 | (58%) | 7 | (58%) | 5 | (56%) | 0 | (0%) | 0 | (0%) | 0 | (0%) |

| | | | | | | |
|---|---|---|---|---|---|---|
| Primary endpoint | 5 (9%) | 5 (14%) | 4 (24%) | 8 (15%)* | 8 (26%)* | 7 (41%) |
| [95% CI] | [3%, 20%] | [5%, 29%] | [7%, 50%] | [7%, 28%] | [12%, 45%] | [18%, 67%] |

All entries are No. (%) of participants;
VR = Virological Response;
EOT = End of Treatment
*One participant with peg-IFN EOT VR did not complete all planned study visits and therefore had no data available within the primary endpoint analysis window. No relapse was observed for this participant.
[1] Relapse (while off-treatment) is defined as any elevation of HBsAg or HBV DNA >=LLOQ and results in PE non-response.
[2] Percentages based on “VR at PegIFN EoT”.

In contrast with B-Clear study results, the proportion of participants who achieved the primary outcome was higher with 12-week (Arm 2) than 24-week bepirovirsen treatment (Arm 1). However, the 95% confidence intervals (CIs) were wide and overlapped, indicating a high level of variability and uncertainty regarding between-treatment differences. The observed difference in efficacy between arms appeared to be driven by a higher relapse rate in Arm 1 during the Peg-IFN treatment window and during off-treatment follow-up. Arm 1 was disproportionately impacted by COVID-19 lockdowns during the Peg-IFN eligibility and treatment periods.

A logistic regression model was constructed to determine variables associated with primary efficacy response success using a forward stepwise selection approach by selecting from the following ordered variables based on the AIC: age, race, sex, BMI, fibrosis score, length of diagnosis, type of NA used at baseline, ALT, urine albumin, prior interferon use and GFR. The final subgroup logistic regression model identified a few variables as statistically significantly associated with response, including low baseline HBsAg level, high body mass index, and treatment with tenofovir alafenamide fumarate or tenofovir disoproxil fumarate (Table 3).

TABLE 3
Logistic Regression of Variables Potentially
Associated with Response.

| | Proportion of | | |
|---|---|---|---|
| | participants, | Odd ratio | p |
| Parameter | n (%) | (95% CI) | value |
| Arms | | | |
| Arm 1 (reference) | 5/54 (9.3) | 1.01 (0.17, 5.96) | 0.99 |
| Arm 2 | 8/53 (15.1) | | |
| Baseline HBsAg | | | |
| ≤1000 IU/mL | 11/34 (32.4) | 0.05 (0.00, 0.26) | <0.0001 |
| (reference) | | | |
| >1000 IU/mL | 2/73 (2.7) | | |
| Baseline HBeAg status | | | |
| Negative | 12/79 (15.2) | 0.28 (0.01, 3.27) | 0.3263 |
| (reference) | | | |
| Positive | 1/28 (3.6) | | |
| Age | NA/107 | 1.05 (0.97, 3.27) | 0.2580 |
| Race | | | |
| White (reference) | 10/46 (21.7) | | |
| Asian | 3/56 (5.4) | 0.43 (0.06, 3.14) | 0.3884 |
| Black or African | 0/5 (0) | 0.01 (<0.01, 1.09) | 0.0539 |
| American | | | |
| BMI (kg/m²) | NA/107 | 1.34 (1.05, 1.93) | 0.0183 |
| Duration of HBV | NA/107 | 1.03 (0.97, 1.10) | 0.2928 |
| infection | | | |
| Type of NA | | | |
| TAF or TDF | 10/70 (14.3) | | |
| (reference) | | | |
| Entecavir | 2/31 (6.5) | 0.09 (0.01, 0.61) | 0.0119 |
| Other | 1/6 (16.7) | 0.95 (0.06, 11.66) | 0.9703 |
| ALT (IU/mL) | NA/107 | 0.95 (0.87, 1.01) | 0.0927 |
| Urine Albumin (mg/L) | NA/107 | 0.99 (0.91, 1.09) | 0.8125 |

ALT, alanine transaminase;
BMI, body mass index;
HBeAg, hepatitis B e antigen;
HBsAg, hepatitis B surface antigen;
HBV, hepatitis B virus;
NA, nucleos(t)ide analogue;
TAF, Tenofovir alafenamide fumarate;
TFD, Tenofovir disoproxil fumarate

The B-Together trial results show sequential therapy with bepirovirsen followed by Peg-IFN is tolerable and effective in participants with chronic HBV infection on stable NA. Participants had a similar response to bepirovirsen treatment as seen in B-Clear, with increased response rates in participants with lower baseline HBsAg. Addition of Peg-IFN treatment improved the off-treatment maintenance of the bepirovirsen response by preventing relapse. These data support use of Peg-IFN treatment, in participants who have responded to bepirovirsen to maintain HBsAg and HBV DNA loss. These data support that bepirovirsen could be of benefit in combination with other therapies. Overall, this trial highlights sequential therapy as a valid treatment strategy for finite treatment regimens for HBV infection.

### Example 2

### Model-Based Meta Analysis to Predict the Response Rate (HBsAg Loss) for PegIFN-Based Regimens in Specific Chronic Hepatitis B Patient Populations

A model-based meta-analysis (MBMA) was developed to establish absolute effect model of HBsAg loss for PegIFN-based regimens that can be leveraged to perform simulations that predict response rate (HBsAg loss) for given populations. A systematic review of clinical trials and prospective and retrospective cohorts investigating HBsAg loss with PegIFN treatment with data on baseline HBsAg level published between 2000 and 2022 was conducted. Data used in the MBMA was based on aggregated summary level data from a total of 83 study-strata-arms (63 study strata from randomized controlled trials) producing HBsAg loss results from 13,235 participants at end of treatment. Covariates predicting HBsAg loss in patients treated with PegIFN were treatment duration, baseline HBsAg level and baseline HBeAg positive status. Additional covariates were assessed, and either were not significant or lacked sufficient data.

Table 4 presents the data of 100,000 simulated trials based on different patient characteristics with regards to baseline HBsAg and HBeAg. HBeAg status (% positive) and baseline HBsAg levels (3.09 and 2.73 log 10 IU/mL) are presented to allow comparison to the B-Together results as these represent the proportion of HBeAg positive patients and the median baseline HBsAg levels for the baseline HBsAg≤3000 and ≤1000 IU/mL patient populations, respectively.

TABLE 4
Model-Based Meta Analysis Results for PegIFN-based Regimens

| | | Baseline | PegIFN | |
|---|---|---|---|---|
| PegIFN | | HBsAg | Monotherapy | PegIFN + NA |
| Treatment | HBeAg | (log10 | % HBsAg loss | % HBsAg loss |
| Duration | Status | IU/mL) | (95% CI) | (95% CI) |
| 48 Weeks | 23.5% | 3.09 | 9.07 (2.41-28.6) | 12.4 (3.37-36.6) |
| | positive | | | |
| 48 Weeks | 11.8% | 2.73 | 13.7 (3.72-39.5) | 18.3 (5.12-48.5) |
| | positive | | | |

### Example 3

Phase 3 Study to Assess the Efficacy and Safety of Treatment with Bepirovirsen and PegIFN Sequential Therapy in Nucleos(t)ide Analogue-Treated Participants with Chronic Hepatitis B Virus (B-Sustained).

### A. Study Design

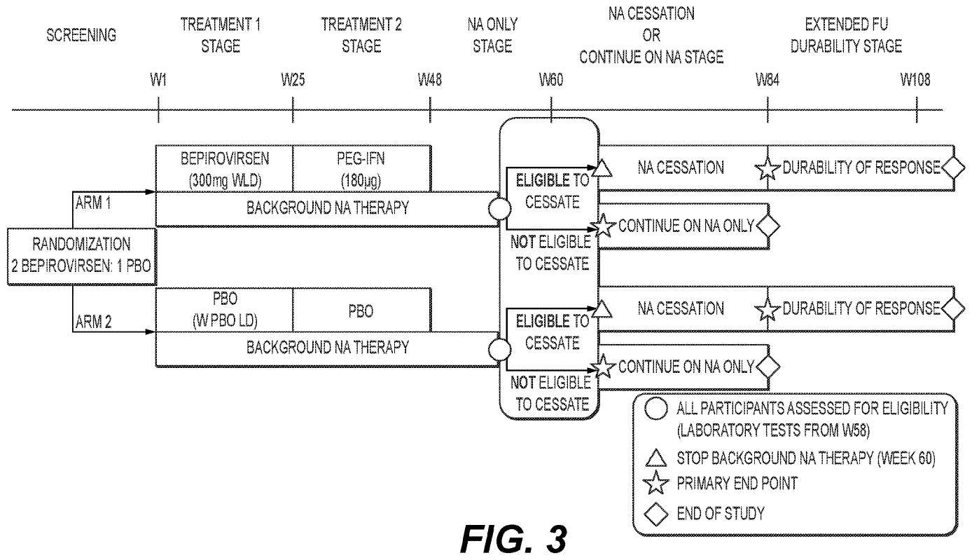

This is a Phase 3b, multicenter, randomized, double-blind, study to assess the efficacy, safety, PK profile, and the durability of HBsAg suppression observed with sequential therapy with bepirovirsen 300 mg weekly for 24 weeks (with 2 loading doses) followed by PegIFN 180 μg weekly for 24 weeks as compared with 48 weeks treatment with placebo control in participants with chronic HBV infection on background NA treatment with baseline HBsAg levels ≤3000 IU/mL (participants stratified by HBsAg levels) (See FIG. 3).

### Study Details:

The study duration may be up to 117 weeks (includes 45 to 60 day screening).
The study has 5 stages:
   Investigational product treatment stage, which is split into 2 stages:
      Stage 1=Investigational product treatment stage 1 (TS1) with bepirovirsen (or placebo) on background NA treatment.
      Stage 2=Investigational product treatment stage 2 (TS2) with PegIFN (or placebo) on background NA treatment.
   Stage 3=NA only.
   Stage 4=NA cessation or continue with NA.
   Stage 5=Extended follow up and durability of response.
The investigational product treatment stage will be up to 48 weeks to total, TS1 and TS2 will each be 24 weeks in duration.
There are 2 arms in the study and participants will be randomised to either arm 1 or arm 2 of the study:
Arm 1:
   TS1 will be bepirovirsen 300 mg weekly for 24 weeks (with 2 loading doses)
   TS2 which will be PegIFN 180 μg weekly for 24 weeks.
Arm 2:
   TS1 will be placebo weekly for 24 weeks (with 2 loading doses)
   TS2 which will be placebo weekly for 24 weeks.
During stage 3 (NA only) background NA will continue for 12 weeks.
At week 60, participants will be assessed for their eligibility to stop background NA.
   Participants that meet the eligibility criteria for NA cessation can stop NA.
      these participants will be followed for an additional 48 weeks, which will include:
         24 weeks NA cessation (stage 4)
         24 weeks follow-up and durability of response (stage 5).
   For participants that are eligible to stop NA the study duration will be up to approximately 117 weeks.
   Participants that do not meet eligibility criteria for NA cessation will continue on NA therapy for 24 weeks and will reach end of study (study duration will be up to approximately 93 weeks).
The visit frequency will decrease as the study progresses. Dosing visits will be approximately weekly (up to Week 24), then approximately biweekly to bimonthly during treatment stage 2 (Weeks 25 to Week 48), then approximately weekly to biweekly (between Weeks 48 to Week 60) and then tapering off to approximately monthly for the duration of the final durations of the study. In select countries/sites, participants may have the option to use a centralized home nursing provider for most visits.

### B. Objectives and Estimands

Objectives, endpoints and estimands are as follows.

| | |
|---|---|
| Objective(s) | Endpoint(s) |

Primary

| | |
|---|---|
| Efficacy: To assess the sequential | Functional cure (FC) 24 weeks after |
| treatment effect of 24 weeks bepirovirsen | discontinuation of all chronic HBV |
| (with loading doses) followed by 24 weeks | treatment (bepirovirsen/placebo, |
| PegIFN compared to placebo to achieve | PegIFN/placebo and NA) in the absence |
| functional cure in participants with chronic | of rescue medication. |
| HBV infection on NA treatment with | |
| baseline HBsAg ≤3000 IU/mL. | |

Secondary

| | |
|---|---|
| Efficacy: To assess the sequential | Functional cure 24 weeks after |
| treatment effect of 24 weeks bepirovirsen | discontinuation of all chronic HBV |
| (with loading doses) followed by 24 weeks | treatment (bepirovirsen/placebo, |
| PegIFN compared to placebo to achieve | PegIFN/placebo and NA) in the absence |
| functional cure in participants with chronic | of rescue medication. |
| HBV infection on NA treatment with | |
| baseline HBsAg ≤1000 IU/mL. | |
| Efficacy: To assess the sequential | Sustained suppression of HBV DNA |
| treatment effect of 24 weeks bepirovirsen | (<lower limit of quantification [LLOQ]) |
| (with loading doses) followed by 24 weeks | 24 weeks after discontinuation of all |
| PegIFN compared to placebo on HBV | chronic HBV treatment |
| DNA suppression off-treatment after a | (bepirovirsen/placebo, PegIFN/placebo |
| finite duration of therapy in participants | and NA) in the absence of rescue |
| with chronic HBV infection on NA | medication. |
| treatment with baseline HBsAg ≤3000 IU/mL | |
| Efficacy: To assess the sequential | Sustained suppression of HBV DNA |
| treatment effect of 24 weeks bepirovirsen | (<LLOQ) 24 weeks after discontinuation |
| (with loading doses) followed by 24 weeks | of all chronic HBV treatment |
| PegIFN compared to placebo on HBV | (bepirovirsen/placebo, PegIFN/placebo |
| DNA suppression off-treatment after a | and NA) in the absence of rescue |
| finite duration of therapy in participants | medication. |
| with chronic HBV infection on NA | |
| treatment with baseline HBsAg ≤1000 IU/mL. | |

Safety

| | |
|---|---|
| Safety: To assess the safety and | Occurrences of adverse events (AEs) |
| tolerability of 24 weeks treatment with | and serious adverse event (SAEs). |
| bepirovirsen (with loading doses) followed | Change from baseline in clinical |
| by 24 weeks PegIFN in participants with | laboratory tests and vital signs. |
| chronic HBV infection on NA treatment. | |

Definition of Functional Cure: Sustained suppression (24 weeks of HBV DNA (<LLOQ) off all HBV Treatment and HBsAg not detected with or without HBsAb after a finite duration of therapy.

Unless otherwise specified, all primary, secondary, and exploratory study objectives will be assessed using estimands defined with the following common elements in terms of population, treatment comparison and approach for managing intercurrent events. Assessment of eligibility for NA cessation and response after discontinuation of all HBV (or study) treatments will be based on the analysis window.

The primary clinical question of interest is to assess if bepirovirsen+pegIFN sequential therapy increases FC response rate in the absence of rescue medication in participants with chronic HBV infection on NA treatment with HBsAg≤3000 IU/mL at baseline as compared to placebo, regardless of discontinuation of, interruption in, or non-adherence to bepirovirsen, pegIFN or NA and regardless of non-rescue medication including medication taken with proven anti-HBV activity not for the management of virologic breakthrough.

### Primary Estimand

| | |
|---|---|
| Population | Participants with chronic HBV infection on NA treatment with |
| | HBsAg ≤3000 IU/mL at baseline |
| Treatment | Arm 1: Sequential therapy of: 24 weeks of bepirovirsen 300 mg |
| | once/week (including 2 loading doses) + NA treatment followed by |
| | 24 weeks of pegIFN 180 mcg once/week + NA treatment followed |
| | by 12 weeks of NA treatment only. |
| | Arm 2: Placebo (including 2 loading doses) weekly for 48 weeks + |
| | NA treatment followed by 12 weeks of NA treatment only. |
| Endpoint | Functional cure 24 weeks after discontinuation of all chronic HBV |
| | treatment (bepirovirsen, PegIFN, and NA) in the absence of rescue |
| | medication. |
| Intercurrent | The use of rescue medication has been incorporated into the |
| events | definition of variable, as functional cure can only be achieved |
| | in the absence of rescue medication (composite strategy). |
| | The intercurrent events of discontinuation of, interruption in, or |
| | non-adherence to bepirovirsen, pegIFN or NA will be ignored |
| | (treatment policy strategy). |
| | The intercurrent events of medication taken with proven anti-HBV |
| | activity not for the management of virologic breakthrough will be |
| | ignored (treatment policy strategy) |
| Population | Common risk difference of the proportion of participants who |
| Summary | achieve FC between bepirovirsen + PegIFN treatment arm and |
| | placebo arm. |

### Key Secondary Estimands

Treatment and intercurrent events are defined the same in secondary estimands as in the primary estimand. Populations and population summaries are defined for 3 secondary objectives as below:

### Key Secondary Efficacy Objective:

To assess the treatment effect of sequential therapy Bepirovirsen+pegIFN sequential therapy in participants with chronic HBV infection on NA treatment with baseline HBsAg ≤1000 IU/mL.

The clinical question of interest is to assess if Bepirovirsen+pegIFN sequential therapy increases FC in the absence of rescue medication in participants with chronic HBV infection on NA treatment with HBsAg≤1000 IU/mL at baseline as compared to placebo regardless of discontinuation of, interruption in, or non-adherence to bepirovirsen or NA and regardless of non-rescue medication including medication taken with proven anti-HBV activity not for the management of virologic breakthrough.

| | |
|---|---|
| Population | Participants with chronic HBV infection on NA |
| | treatment with HBsAg ≤1000 IU/mL at baseline |
| Endpoint | Functional cure 24 weeks after discontinuation |
| | of all chronic HBV treatment (bepirovirsen, PegIFN, |
| | and NA) in the absence of rescue medication. |
| Population | Difference in proportion of participants who |
| Summary | achieve FC between the bepirovirsen + PegIFN |
| | treatment arm and placebo arm. |

### Key Secondary Efficacy Objective:

To assess the treatment effect of bepirovirsen+pegIFN sequential therapy treatment on HBV DNA suppression off-treatment after a finite duration of therapy in participants with chronic HBV infection on NA treatment with baseline HBsAg≤3000 IU/mL.

The clinical question of interest is to assess if bepirovirsen+pegIFN sequential therapy improves HBV DNA suppression off-treatment after a finite duration of therapy in the absence of rescue medication in participants with chronic HBV infection on NA treatment with HBsAg ≤3000 IU/mL at baseline as compared to placebo, regardless of discontinuation of, interruption in, or non-adherence to bepirovirsen, pegIFN or NA and regardless of non-rescue medication including medication taken with proven anti-HBV activity not for the management of virologic breakthrough.

| | |
|---|---|
| Population | Participants with chronic HBV infection on NA |
| | treatment with HBsAg ≤3000 IU/mL at baseline. |
| Endpoint | Sustained suppression (24 weeks) of HBV DNA |
| | (<LLOQ) after discontinuation of all chronic |
| | HBV treatment (bepirovirsen, PegIFN, and NA) |
| | in the absence of rescue medication. |
| Population | Difference in proportion of participants who |
| Summary | achieve NA cessation and sustained suppression |
| | of HBV DNA (<LLOQ) off treatment between the |
| | bepirovirsen + PegIFN treatment arm and |
| | placebo arm. |

### Key Secondary Efficacy Objective:

To assess the treatment effect of sequential therapy of bepirovirsen+pegIFN sequential therapy in HBV DNA suppression off-treatment after a finite duration of therapy in participants with chronic HBV infection on NA treatment with baseline HBsAg≤1000 IU/mL.

The clinical question of interest is to assess if bepirovirsen+pegIFN sequential therapy improves HBV DNA suppression off-treatment after a finite duration of therapy in the absence of rescue medication in participants with chronic HBV infection on NA treatment with HBsAg ≤1000 IU/mL at baseline as compared to placebo, regardless of discontinuation of, interruption in, or non-adherence to bepirovirsen, pegIFN or NA and regardless of non-rescue medication including medication taken with proven anti-HBV activity not for the management of virologic breakthrough.

| | |
|---|---|
| Population | Participants with chronic HBV infection on NA |
| | treatment with HBsAg ≤1000 IU/mL at baseline. |
| Endpoint | Sustained suppression (24 weeks) of HBV DNA |
| | (<LLOQ) after discontinuation of all chronic |
| | HBV treatment (bepirovirsen, PegIFN, and NA) |
| | in the absence of rescue medication. |
| Population | Common risk difference in proportion of participants |
| Summary | who achieve NA cessation and sustained suppression |
| | of HBV DNA (<LLOQ) between the bepirovirsen + |
| | PegIFN treatment arm and placebo arm. |

### Additional Estimands

Additional estimands are defined to support the primary and key secondary estimands with alternative strategies for intercurrent events.

The first additional estimand for the primary and key secondary objectives address the clinical question of whether bepirovirsen+pegIFN sequential therapy increases FC and improves HBV DNA suppression in the absence of rescue medication as compared to placebo regardless of rescue medication use and had no discontinuation of, interruption in, and non-adherence to Investigational Product caused by wide disruptive events and resulting in a gap of ≥21 days between doses occurred. The attributes for this additional estimand are the same as for the primary estimand except a hypothetical strategy will be used for the intercurrent events of discontinuation of, interruption in, and non-adherence to Investigational Product caused by wide disruptive events and resulting in a gap of ≥21 days between doses.

The second additional estimand for the primary and key secondary objectives address the clinical question of bepirovirsen+pegIFN sequential therapy increases FC and improves HBV DNA suppression in the absence of rescue medication as compared to placebo regardless of discontinuation of, interruption in, and non-adherence to Investigational Product, had no use of medication with proven anti-HBV activity not for the management of HBV DNA reversion occurred. The attributes for this additional estimand are the same as for the primary estimand except a hypothetical strategy will be used for the intercurrent event of taking medication with proven anti-HBV activity not for the management of HBV DNA reversion.

### Exploratory Estimands:

Assessment of eligibility for NA cessation and response after discontinuation of all HBV (or study) treatments will be based on the predefined analysis window.

| | |
|---|---|
| Population | Separate assessments for the following: |
| | Participants with chronic HBV infection, On-NA, and HBsAg ≤1000 |
| | IU/mL at baseline |
| | Participants with chronic HBV infection, On-NA, and HBsAg ≤3000 |
| | IU/mL at baseline. |
| Endpoints | Categorical Variables: |
| | Achieving HBsAg and HBV DNA <LLOQ at Week 48 |
| | Meeting NA cessation criteria at Week 60 |
| | Meeting NA cessation criteria at Week 60 and sustaining FC for |
| | a further 48 weeks |
| | Meeting NA cessation criteria at Week 60 and sustaining HBV |
| | DNA <LLOQ for a further 48 weeks |
| | HBeAg status at Weeks 12, 24, 37, 48, 60, 84 and 108 |
| | Anti-HBeAg status at Weeks 12, 24, 37, 48, 60, 84, and 108. |
| | Continuous Variables: |
| | Actual values and change from baseline over time for HBsAg, |
| | HBV DNA, and HBs antibody (anti-HBsAg) |
| | Relationship between FC and on treatment biomarkers including |
| | but not limited to: on treatment HBsAg and change from baseline. |
| Treatments | Sequential therapy of bepirovirsen 300 mg weekly (including |
| | 2 loading doses) + NA treatment for 24 weeks followed by |
| | PegIFN 180 μg weekly + NA treatment for 24 weeks followed |
| | by NA treatment until NA cessation criteria is met. |
| | Placebo weekly for 48 weeks + NA treatment followed by NA |
| | treatment until NA cessation criteria is met. |
| Intercurrent | For the categorical variables of achieving HBsAg and HBV |
| events | DNA <LLOQ at Week 24 and anti-HBeAg status at Weeks 12, |
| | 24, 37, 48, 60, 84, and 108, data after the use of rescue |
| | medication will be analysed as collected (treatment policy |
| | strategy) |
| | For the continuous variables of actual values and change from |
| | baseline over time for HBsAg, HBV DNA, and anti-HBsAg, |
| | data after the use of rescue medication will be analysed as |
| | collected (treatment policy strategy) |
| | For the categorical variable of meeting NA cessation criteria at |
| | Week 60, meeting NA cessation criteria at Week 60 and |
| | sustaining FC for a further 48 weeks, meeting NA cessation |
| | criteria at Week 60 and sustaining HBV DNA <LLOQ for a |
| | further 60 weeks, the use of rescue medication has been |
| | incorporated into the definition of variables (composite strategy) |
| | For the continuous variable of relationship between FC and on |
| | treatment biomarkers, the use of rescue medication has been |
| | incorporated into the definition of variables (composite strategy) |
| | For discontinuation of, interruption in, or non-adherence to |
| | Bepirovirsen, PegIFN, or NA regardless of cause, the data will |
| | be analysed as collected (treatment policy strategy). |
| Population | Categorical variables: Proportion of participants in each |
| Summary | category for each treatment arm |
| | Continuous variables: Mean values and mean changes from |
| | baseline for each variable in each treatment arm. |

### C. Study Population: Inclusions and Exclusion Criteria

### Inclusion Criteria Include:

### Age

1. At least 18 years of age at the time of signing the informed consent (if country/site age requirements for consent differ, the more stringent [e.g., higher age] restriction will be required for that country/site).

### Type of Participant and Disease Characteristics

2. Participants who are HBeAg negative or HBeAg-positive at Screening.
3. Participants who have documented chronic HBV infection >6 months prior to Screening AND
   Currently receiving stable NA therapy (except Telbivudine) defined as no changes to their NA regimen from at least 6 months prior to Screening and with no planned changes to the stable regimen over the duration of the study.
4. Plasma or serum HBsAg concentration >100 IU/mL and HBsAg concentration ≤3000 IU/mL.
5. Plasma or serum HBV DNA concentration must be adequately suppressed, defined as plasma or serum HBV DNA <90 IU/mL.
6. Alanine aminotransferase (ALT)≤2×ULN.
7. Participants who are willing and eligible to be treated with PegIFN.
8. Participants who are willing and able to cease their NA treatment in accordance with the protocol.

### Informed Consent

9. Capable of giving signed informed consent, which includes compliance with the requirements and restrictions listed in the ICF and in this protocol.

### Exclusion Criteria Include:

1. Clinically significant abnormalities, aside from chronic HBV infection in medical history (e.g., moderate severe liver disease other than chronic HBV, acute coronary syndrome within 6 months of screening, major surgery within 3 months of screening, significant/unstable cardiac disease, uncontrolled diabetes, bleeding diathesis or coagulopathy) or physical examination.
2. Co-infection with:
   a. Hepatitis C virus, including participants with a past infection that has been cured for <12 months at the time of screening (defined as having been HCV RNA negative for 12 months).
   b. Hepatitis D virus, defined as HDV antibody positive.
   c. Human immunodeficiency virus (HIV).
3. History of or suspected liver cirrhosis and/or evidence of cirrhosis as determined by:
   a. Both APRI >2 and FibroSure/FibroTest result >0.7
      If only 1 parameter (APRI or FibroSure/FibroTest) result is positive, a discussion with the Medical Monitor is required before inclusion in study is permitted.
   b. Regardless of APRI or Fibrosure/Fibro Test score, if the participant has historic record of either/both liver biopsy or liver stiffness measurements in their medical records and meets 1 of the following criteria, they will be excluded from the study:
      Liver biopsy (i.e., Metavir Score F4)
      Liver stiffness >12 kPa.
4. Diagnosed or suspected hepatocellular carcinoma as evidenced by the following:
   a. Alpha fetoprotein concentration >200 ng/ml
   b. If the screening alpha fetoprotein concentration is ≥50 ng/ml and <200 ng/ml, the absence of liver mass must be documented by imaging within 6 months before randomization.
5. History of malignancy within the past 5 years with the exception of specific cancers that are cured by surgical resection (e.g., skin cancer). Participants under evaluation for possible malignancy are not eligible.
6. History of vasculitis or presence of symptoms and signs of potential vasculitis (e.g., vasculitic rash, skin ulceration, repeated blood detected in urine without identified cause) current or history of autoimmune conditions or history/presence of other diseases that may be associated with vasculitis condition (e.g., systemic lupus erythematosus, rheumatoid arthritis, relapsing polychondritis, mononeuritis multiplex).
7. History of extrahepatic disorders possibly related to HBV immune conditions (e.g., nephrotic syndrome, any type of glomerulonephritis, polyarteritis nodosa, cryoglobulinemia, uncontrolled hypertension).
8. Poorly controlled thyroid dysfunction or abnormal thyroid stimulating hormone (TSH) levels.
9. Pre-existing severe psychiatric condition or a history of severe psychiatric disorders, including severe depression, suicidal ideation and attempted suicide.
10. History of alcohol or drug abuse/dependence:
   a. Current alcohol use as judged by investigator to potentially interfere with participant compliance.
   b. History of or current drug abuse/dependence as judged by the investigator to potentially interfere with participant compliance.

### Prior/Concomitant Therapy

11. Currently taking, or took within 3 months of screening, any immunosuppressing drugs (e.g., prednisone), other than a short course of therapy (≤2 weeks) or topical/inhaled steroid use.
12. Participants to whom immunosuppressive treatment, including to whom therapeutic doses of steroids is contraindicated, should not be considered for enrolment in the study.
13. Currently taking, or HAS TAKEN within 3 years of Screening, any interferon containing therapy.
14. Participants requiring anti coagulation therapies (e.g., warfarin, Factor Xa inhibitors) or anti-platelet agents (like clopidogrel or aspirin) unless treatment can safely be discontinued throughout duration of the study, by the discretion of the investigator. Occasional use is permitted.
15. The participant has participated in a clinical trial and has received an investigational product within the following time period prior to the first dosing day in the current study: 5 half-lives (if known) or twice the duration (if known) of the biological effect of the study treatment (whichever is longer) or 90 days (if half-life or duration is unknown).
16. Prior treatment with any oligonucleotide or siRNA within 12 months prior to the first dosing day.
17. Prior treatment with bepirovirsen.
18. Participants currently taking, or took within 6 months of screening, telbivudine.

### Diagnostic Assessments

19. Fridericia's QT correction formula (QTcF) ≥450 msec (if single ECG at screening shows QTcF 3450 msec, a mean of triplicate measurements should be used to confirm that participant meets exclusion criterion).
20. Laboratory results as follows:
   a. Platelet count <140×10⁹/L.
   b. Total bilirubin >1.25×ULN.
      For participants with benign unconjugated hyperbilirubinemia with total bilirubin >1.25×ULN, discussion for inclusion to the study is required with the Medical Monitor.
   c. INR>1.25.
   d. Serum albumin ≤3.5 g/dL.
   e. Glomerular filtration rate (GFR)≤60 mL/min/1.73 m2 as calculated by the CKD EPI formula (for Japan, JSN CKDI equation).
   f. Urine albumin to creatinine ratio (uACR)>0.3 mg/mg (or >300 mg/g). In the event of an uACR above this threshold, eligibility may be confirmed by a second measurement.

### Other Exclusions

21. History of/sensitivity to bepirovirsen or peg-IFN containing therapies, or components thereof, or a history of drug or other allergy that, in the opinion of the investigator or Medical Monitor, contraindicates their participation.
22. Participants who do not wish to discontinue taking NA therapy for their chronic HBV infection.

One skilled in the art will readily appreciate that the present disclosure is well adapted to carry out the objects and obtain the ends and advantages mentioned, as well as those inherent therein. The particular embodiments described herein are intended to be representative and exemplary and are not intended as limitations on the scope of the invention. Changes therein and other uses will be apparent to those skilled in the art which are encompassed within the spirit of the invention as defined by the scope of the claims.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

That which is claimed:

1. A method for treating chronic hepatitis B in a human in need thereof, the method comprising administering to the human a therapeutically effective amount of an antisense oligonucleotide (ASO) and a therapeutically effective amount of interferon, wherein the human has a HBsAg baseline level of not greater than a threshold level.

2. (canceled)

3. (canceled)

4. The method of claim 1, wherein the threshold level is in the range of about 1000 IU/mL to 3000 IU/mL.

5. (canceled)

6. The method of claim 1, wherein the threshold level is about 3000 IU/mL.

7. (canceled)

8. (canceled)

9. The method of claim 1, wherein the ASO is administered by subcutaneous injection.

10. The method of claim 1, wherein the ASO is administered at a dose of about 150 mg or 300 mg once weekly.

11. The method of claim 1, wherein the ASO is administered for 12 weeks to 48 weeks.

12. The method of claim 10, wherein the ASO is administered weekly with additional loading doses in the first two weeks on Day 4 and Day 11 following the first dose.

13. (canceled)

14. The method of claim 1, wherein the ASO is administered at a dose of about 300 mg once weekly for 24 weeks.

15. The method of claim 1, wherein interferon is interferon alpha.

16. (canceled)

17. The method of claim 15, wherein interferon alpha is pegylated human interferon alpha-2a.

18. The method of claim 17, wherein PegIFN is administered at a dose of about 180 μg once weekly.

19. (canceled)

20. The method of claim 1, wherein interferon is administered following cessation of the ASO.

21. The method of claim 1, wherein interferon is administered for 12 weeks to 24 weeks following cessation of the ASO.

22. The method of claim 1, wherein the human is on stable nucleoside or nucleotide analogue (NA) therapy.

23. (canceled)

24. (canceled)

25. (canceled)

26. The method of claim 1, wherein the human is HBeAg negative prior to the ASO treatment.

27. The method of claim 1, wherein the human is HBeAg positive prior to the ASO treatment.

28. The method of claim 1, wherein the human's HBV DNA and HBsAg levels are below the lower limit of quantification (LLOQ) 24 weeks after stopping all HBV therapy.

29. (canceled)

30. (canceled)

31. (canceled)

32. (canceled)

33. The method of claim 1, wherein the ASO comprises 20 linked nucleosides and has a nucleobase sequence of SEQ ID NO:1.

34. The method of claim 1, wherein the ASO consists of 20 linked nucleosides and has a nucleobase sequence of SEQ ID NO:1.

35. The method of claim 33, wherein the ASO comprises:
a gap segment consisting of linked deoxynucleosides,
a 5′ wing segment consisting of linked nucleosides, and
a 3′ wing segment consisting of linked nucleosides,
wherein the gap segment is positioned between the 5′ wing segment and the 3′ wing segment and wherein each nucleoside of each wing segment comprises a modified sugar.

36. The method of claim 35, wherein the modified sugar comprises a 2′-O-methoxyethyl(2′-MOE) group or is a bicyclic sugar.

37. (canceled)

38. (canceled)

39. The method of claim 1, wherein the ASO is bepirovirsen.

40. The method of claim 1, wherein the ASO is AUS1493.

41. (canceled)


SEQUENCE LISTING (1 sequence)

SEQ ID NO: 1 - DNA, 20 bp, synthetic construct
  source 1..20: mol_type = other DNA
gcagaggtgaagcgaagtgc